(19) United States
(12) Patent Application Publication
(10) Pub. No.: US 2024/0144045 A1
(43) Pub. Date: May 2, 2024

(54) SEARCH METHOD, SEARCH SYSTEM, RECORDING MEDIUM, PREDICTION MODEL CONSTRUCTION METHOD, AND PREDICTION MODEL CONSTRUCTION DEVICE

(71) Applicant: Panasonic Intellectual Property Management Co., Ltd., Osaka (JP)

(72) Inventors: KEI AMII, Osaka (JP); MASAKI OKOSHI, Kyoto (JP); MIKIYA FUJII, Osaka (JP)

(21) Appl. No.: 18/408,653

(22) Filed: Jan. 10, 2024

Related U.S. Application Data
(63) Continuation of application No. PCT/JP2022/027343, filed on Jul. 12, 2022.

(30) Foreign Application Priority Data
Jul. 27, 2021 (JP) 2021-122609

(51) Int. Cl. G06N 5/022

(52) U.S. Cl. CPC G06N 5/022

(57) ABSTRACT

A computer performs a first step of acquiring initial structures that are atomic arrangement structures in a three-dimensional space which a composition of a material can take, a second step of calculating first energy corresponding to structurally optimized atomic arrangement structures by performing structure optimization on one or some initial structures, a third step of predicting second energy corresponding to atomic arrangement structures obtained in a case where structure optimization is performed on other initial structure(s) by using a prediction model for the other initial structure(s), a fourth step of extracting third energy indicative of a minimum value on the basis of the first energy and the second energy, and a fifth step of outputting the third energy, a first structure, which is an atomic arrangement structure corresponding to the third energy, or the third energy and the first structure.

### BACKGROUND

### 1. Technical Field

The present disclosure relates to a search method for searching for a stable atomic arrangement structure concerning a composition of a material, and others.

### 2. Description of the Related Art

Conventionally, a structure optimizing technique of finding a more stable atomic arrangement structure by ab initio calculation has been developed (see, for example, Jensen, F. (2007). Introduction to computational chemistry. John wiley & sons, 383-389 (hereinafter referred to as Non Patent Literature 1).

Chen, C., Ye, W., Zuo, Y., Zheng, C., & Ong, S. P. (2019). Graph networks as a universal machine learning framework for molecules and crystals. Chemistry of Materials, 31 (9), 3564-3572 (hereinafter referred to as Non Patent Literature 2) discloses a method of estimating a characteristic value such as energy by using machine learning in response to input of an atomic arrangement structure.

### SUMMARY

One non-limiting and exemplary embodiment provides a search method that can efficiently search for a stable atomic arrangement structure concerning a composition of a material, and others.

In one general aspect, the techniques disclosed here feature a search method for searching for a stable atomic arrangement structure in a three-dimensional space concerning a composition of a material, including causing a computer to acquire initial structures that are atomic arrangement structures in the three-dimensional space which the composition of the material can take; calculate first energy corresponding to structurally optimized atomic arrangement structures by performing structure optimization on one or some initial structures among the initial structures; predict second energy corresponding to atomic arrangement structures obtained in a case where structure optimization is performed on other initial structure(s) among the initial structures by using a prediction model for the other initial structure(s); extract third energy indicative of a minimum value on a basis of the first energy and the second energy; and output the third energy, a first structure, which is an atomic arrangement structure corresponding to the third energy, or the third energy and the first structure, in which the prediction model is trained by machine learning to output, in response to input of any atomic arrangement structure, energy corresponding to a structure obtained in a case where structure optimization is performed on the atomic arrangement structure as the second energy.

According to the present disclosure, it is possible to efficiently search for a stable atomic arrangement structure concerning a composition of a material.

It should be noted that general or specific embodiments may be implemented as a device, a system, an integrated circuit, a computer program, a computer-readable recording medium, or any selective combination thereof. Examples of the computer-readable recording medium include non-volatile recording medium such as a compact disc-read only memory (CD-ROM).

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a block diagram illustrating an overall configuration including a search system according to Embodiment 1;

FIG. 2 illustrates an example of data stored in a material database according to Embodiment 1;

FIGS. 3A and 3B illustrate an example of a process of generating initial structures by a generation unit according to Embodiment 1;

FIGS. 4A and 4B illustrate an example of a process of generating initial structures by the generation unit according to Embodiment 1;

FIGS. 5A and 5B illustrate an example of a process of generating initial structures by the generation unit according to Embodiment 1;

FIGS. 6A to 6C illustrate an example of initial structures generated by the generation unit according to Embodiment 1;

FIG. 7A illustrates an example of steric arrangement of an initial structure generated by the generation unit according to Embodiment 1;

FIG. 7B illustrates another example of steric arrangement of an initial structure generated by the generation unit according to Embodiment 1;

FIG. 8 illustrates an example of data stored in a structure storage unit according to Embodiment 1;

FIG. 9 illustrates an example of a process of calculating first energy by a calculation unit according to Embodiment 1;

FIG. 10 illustrates an example of data stored in a calculation result storage unit according to Embodiment 1;

FIG. 11 illustrates an example of a process of performing machine learning of a prediction model by a training unit according to Embodiment 1;

FIG. 12 illustrates an example of a process of predicting second energy by a prediction unit according to Embodiment 1;

FIG. 13 illustrates an example of data generated by a comparing unit according to Embodiment 1;

FIG. 14 illustrates an evaluation example of prediction accuracy of the prediction unit according to Embodiment 1;

FIG. 15 illustrates a result of verification of prediction accuracy of the prediction unit according to Embodiment 1;

FIG. 16 illustrates a result of verification of a correlation between prediction accuracy of the prediction unit and a ratio of data for learning according to Embodiment 1;

FIG. 17 is a flowchart illustrating an operation example of the search system according to Embodiment 1;

FIG. 18 is a block diagram illustrating an overall configuration including a search system according to Embodiment 2;

FIG. 19 illustrates an example of data stored in a calculation result storage unit according to Embodiment 2;

FIG. 20 illustrates an example of a process of performing machine learning of a prediction model by a training unit according to Embodiment 2;

FIG. 21 illustrates a result of verification of prediction accuracy of a prediction unit according to Embodiment 2;

FIG. 22 illustrates a result of verification of a correlation between prediction accuracy of the prediction unit and a ratio of data for learning according to Embodiment 2;

FIG. 23 is a flowchart illustrating an operation example of the search system according to Embodiment 2;

FIG. 24 is a block diagram illustrating an overall configuration including a search system according to Embodiment 3;

FIG. 25 illustrates an example of data generated by a comparing unit according to Embodiment 3;

FIG. 26 is a flowchart illustrating an operation example of the search system according to Embodiment 3;

FIG. 27 is a block diagram illustrating an overall configuration including a search system according to Embodiment 4; and

FIG. 28 is a flowchart illustrating an operation example of the search system according to Embodiment 4.

### DETAILED DESCRIPTIONS

### Underlying Knowledge Forming Basis of the Present Disclosure

In material development, a thermodynamically stable atomic arrangement structure, that is, a stable structure in each substance needs to be found in order to calculate a property such as a thermodynamic property or safety by simulation. The stable atomic arrangement structure can be found by structure optimization. Therefore, structure optimization is used as a tool for analyzing a substance or developing a novel substance. Non Patent Literature 1 discloses a structure optimization method using ab initio calculation.

In order to find a thermodynamically stable atomic arrangement structure in an unknown novel substance, structure optimization is performed on a candidate atomic arrangement structure which the novel substance can take. The candidate atomic arrangement structure can be obtained by partially substituting an atom included in an atomic arrangement structure of a known substance. Therefore, candidate structures can be obtained by changing an atom to be substituted. Structure optimization is performed one or more times on each of the candidate structures, and thus energy, that is, total energy of the structurally-optimized candidate structure is calculated. An atomic arrangement structure corresponding to energy of a smallest value among calculated energy values, that is, a structurally-optimized candidate structure is determined as a thermodynamically most stable atomic arrangement structure in the novel substance.

When the number of atoms that constitute the unknown novel substance, the number of atoms that can be substituted also increases. As a result, the number of candidate structures can become very large, that is, combinatorial explosion can occur. In such a case, there is a problem that if processing of performing structure optimization and calculating energy is performed on all candidate structures, computation takes an enormous time, and it is unrealistic to perform the computation.

In recent years, a method of performing regression or classification in response to input of a graph structure by a graph neural network has been proposed. In this method, a graph structure constituted by nodes and edges representing links between nodes is input, and a correspondence relationship with an output is learned by computation such as convolution.

In particular, Non Patent Literature 2 proposes a graph neural network model that converts atoms into nodes and converts bonds into edges in an atomic arrangement structure concerning a composition of a material and predicts a property value such as energy from the atomic arrangement structure. Non Patent Literature 2 indicates that a model that predicts a material property such as energy from an atomic arrangement structure included in a public database with high accuracy can be constructed by this method.

Note that Non Patent Literature 1 is a conventional art document that discloses a basic technique of structure optimization and does not disclose training a prediction model by machine learning. Non Patent Literature 2 merely discloses a method for predicting a material property from an atomic arrangement structure and does not disclose searching for a stable atomic arrangement structure.

The inventors of the present application noticed that an atomic arrangement structure and energy can be associated by a graph neural network. As a result of studies, the inventors of the present application found a technique that can efficiently search for a thermodynamically stable atomic arrangement structure from among candidate atomic arrangement structures which a composition of a material can take as compared with conventional arts. It has been revealed that this can reduce a computation cost and can search for a stable atomic arrangement structure with high accuracy.

That is, a search method according to an aspect of the present disclosure is a search method for searching for a stable atomic arrangement structure in a three-dimensional space concerning a composition of a material, including causing a computer to: acquire initial structures that are atomic arrangement structures in the three-dimensional space which the composition of the material can take; calculate first energy corresponding to structurally optimized atomic arrangement structures by performing structure optimization on one or some initial structures among the initial structures; predict second energy corresponding to atomic arrangement structures obtained in a case where structure optimization is performed on other initial structure(s) among the initial structures by using a prediction model for the other initial structure(s); extract third energy indicative of a minimum value on a basis of the first energy and the second energy; and output the third energy, a first structure, which is an atomic arrangement structure corresponding to the third energy, or the third energy and the first structure, in which the prediction model is trained by machine learning to output, in response to input of any atomic arrangement structure, energy corresponding to a structure obtained in a case where structure optimization is performed on the atomic arrangement structure as the second energy.

For example, in a case where n initial structures are acquired in the acquiring, the one or some initial structures in the calculating may be m initial structures, and the other initial structure(s) in the predicting may be (n-m) initial structures, n being an integer of two or more and m being an integer that is equal to or more than one and is less than n.

This makes it possible to efficiently search for a stable atomic arrangement structure concerning a composition of a material and makes it easy to reduce a computation cost.

The third energy may be a smallest value among the first energy and the second energy.

This makes it possible to efficiently search for a most stable atomic arrangement structure concerning a composition of a material.

In the acquiring, a known structure that is an atomic arrangement structure in a three-dimensional space of a known material similar to the composition of the material may be acquired, and the initial structures may be generated on the basis of the known structure. For example, the known material may contain at least one different kind of element from an element contained in the composition of the material; and the acquiring may include a process of substituting the different kind of element with a same kind of element as an element contained in the composition of the material. For example, the acquiring may include a process of expanding the known structure in at least a one-dimensional direction.

This makes it easy to generate initial structures by relatively simple processing by using a known structure.

The prediction model may be a model trained by machine learning by using a first learning dataset including the initial structures as input data and including the first energy corresponding to the initial structures as correct answer data.

This makes it easy to predict energy corresponding to a structure obtained in a case where structure optimization is performed on an input initial structure with high accuracy.

The prediction model may be a model trained by machine learning by further using a second learning dataset including the structurally optimized atomic arrangement structures as input data and including the first energy corresponding to the structurally optimized atomic arrangement structures as correct answer data.

This makes it easy to predict energy corresponding to a structure obtained in a case where structure optimization is performed on an input initial structure with higher accuracy.

The number of one or some initial structures in the calculating may be equal to or less than 90% of the total number of initial structures.

This makes it easy to efficiently search for a stable atomic arrangement structure concerning a composition of a material while keeping a computation cost small.

A search system according to one aspect of the present disclosure is a search system for searching for a stable atomic arrangement structure in a three-dimensional space concerning a composition of a material, including: a generator that generates initial structures that are atomic arrangement structures in the three-dimensional space which the composition of the material can take; a calculator that calculates first energy corresponding to structurally optimized atomic arrangement structures by performing structure optimization on one or some initial structures among the initial structures; a predictor that predicts second energy corresponding to atomic arrangement structures obtained in a case where structure optimization is performed on other initial structure(s) among the initial structures by using a prediction model for the other initial structure(s); and an output unit that outputs the first energy and the second energy, in which the prediction model is trained by machine learning to output, in response to input of any atomic arrangement structure, energy corresponding to a structure obtained in a case where structure optimization is performed on the atomic arrangement structure as the second energy.

This makes it possible to efficiently search for a stable atomic arrangement structure concerning a composition of a material and makes it easy to reduce a computation cost.

The output unit may output third energy indicative of a minimum value that is extracted on a basis of the first energy and the second energy, a first structure, which is an atomic arrangement structure corresponding to the third energy, or the third energy and the first structure.

This makes it possible to efficiently search for a most stable atomic arrangement structure concerning a composition of a material.

A recording medium according to an aspect of the present disclosure is a non-volatile computer-readable recording medium storing a program for searching for a stable atomic arrangement structure in a three-dimensional space concerning a composition of a material, the program causing a computer to execute a process including: acquiring initial structures that are atomic arrangement structures in the three-dimensional space which the composition of the material can take; calculating first energy corresponding to structurally optimized atomic arrangement structures by performing structure optimization on one or some initial structures among the initial structures; predicting second energy corresponding to atomic arrangement structures obtained in a case where structure optimization is performed on other initial structure(s) among the initial structures by using a prediction model for the other initial structure(s); outputting the first energy and the second energy, in which the prediction model is trained by machine learning to output, in response to input of any atomic arrangement structure, energy corresponding to a structure obtained in a case where structure optimization is performed on the atomic arrangement structure as the second energy. For example, the program may further cause the computer to execute extracting third energy indicative of a minimum value on the basis of the first energy and the second energy, and in the outputting, the third energy, a first structure, which is an atomic arrangement structure corresponding to the third energy, or the third energy and the first structure may be further output.

This makes it possible to efficiently search for a stable atomic arrangement structure concerning a composition of a material and makes it easy to reduce a computation cost.

A prediction model construction method according to an aspect of the present disclosure includes causing a computer to: acquire an initial structure that is an atomic arrangement structure in a three-dimensional space which a composition of a material can take; and perform machine learning so that in response to input of any atomic arrangement structure, energy corresponding to a structure obtained in a case where structure optimization is performed on the atomic arrangement structure is output by using a learning dataset including the initial structure as input data and including, as correct answer data, energy corresponding to an atomic arrangement structure obtained by performing structure optimization on the initial structure.

This makes it possible to construct a prediction model that can efficiently search for a stable atomic arrangement structure concerning a composition of a material and makes it easy to reduce a computation cost.

A prediction model construction device according to an aspect of the present disclosure includes a generator that generates an initial structure that is an atomic arrangement structure in a three-dimensional space which a composition of a material can take; and a trainer that performs machine learning so that in response to input of any atomic arrangement structure, energy corresponding to a structure obtained in a case where structure optimization is performed on the atomic arrangement structure is output by using a learning dataset including the initial structure as input data and including, as correct answer data, energy corresponding to an atomic arrangement structure obtained by performing structure optimization on the initial structure.

This makes it possible to construct a prediction model that can efficiently search for a stable atomic arrangement structure concerning a composition of a material and makes it easy to reduce a computation cost.

A search method according to an aspect of the present disclosure is a search method for searching for a stable atomic arrangement structure in the three-dimensional space concerning the composition of the material by using a prediction model trained by machine learning by the prediction model construction device, the search method including causing a computer to: acquire the initial structures; predict energy corresponding to atomic arrangement structures obtained in a case where structure optimization is performed on the initial structures by using the prediction model for the initial structures; and extract energy indicative of a minimum value from among predicted values of the energy.

This makes it possible to efficiently search for a stable atomic arrangement structure concerning a composition of a material and makes it easy to reduce a computation cost.

A search method according to an aspect of the present disclosure is a search method for searching for a stable atomic arrangement structure in the three-dimensional space concerning the composition of the material by using a prediction model trained by machine learning by the prediction model construction device, the search method including causing a computer to: acquire the initial structures; calculate first energy corresponding to structurally optimized atomic arrangement structures by performing structure optimization on one or some initial structures among the initial structures; predict second energy corresponding to an atomic arrangement structure obtained in a case where structure optimization is performed on at least one of the one or some initial structures by using the prediction model for the at least one of the one or some initial structures; and verify prediction accuracy of the prediction model by comparing the first energy and the second energy.

This makes it easy to realize a prediction model having sufficient prediction accuracy by verifying prediction accuracy of the prediction model.

The search method may further include causing the computer to: in a case where a result in the verifying satisfies a predetermined condition, predict the second energy corresponding to atomic arrangement structures obtained in a case where structure optimization is performed on other initial structure(s) among the initial structures by using the prediction model for the other initial structure(s); and extract third energy indicative of a minimum value on the basis of the first energy and the second energy.

This makes it easy to more efficiently search for a stable atomic arrangement structure concerning a composition of a material by using a prediction model of relatively high prediction accuracy.

The present disclosure may be realized as a computer program that causes a computer to perform characteristic processing included in the search method or prediction model construction method according to the present disclosure. Needless to say, such a computer program can be distributed by a computer-readable non-transitory recording medium such as a CD-ROM or over a communication network such as the Internet.

Embodiments are specifically described below with reference to the drawings.

Note that the embodiments below illustrate general or specific examples of the present disclosure. Numerical values, shapes, constituent elements, steps, the order of steps, and the like illustrated in the embodiments below are examples and do not limit the present disclosure. Among constituent elements in the embodiments below, constituent elements that are not described in independent claims indicating highest concepts are described as optional constituent elements. Contents in all embodiments can be combined. Each drawing is a schematic view and is not necessarily strict illustration. In each drawing, identical constituent members are given identical reference signs.

A search system according to an embodiment of the present disclosure may be configured so that all constituent elements are included in a single computer or may be configured as a system in which constituent elements are distributed into computers.

### Embodiment 1

### Embodiment 1: Description of Constituents

A search system 100 (search method, or program) according to Embodiment 1 of the present disclosure is described in detail below with reference to the drawings. The search system 100 (search method, or program) according to Embodiment 1 is a system (method, or program) for searching for a stable atomic arrangement structure in a three-dimensional space concerning a composition of a material. The “stable structure” as used herein is such a structure that force acting on each atom included in an atomic arrangement structure (i.e., a crystal structure) is equal to or less than a threshold value and is such a structure that energy (total energy) corresponding to a structure becomes smallest. Note that the threshold value can be set by a user as appropriate and can be a value close to zero. This is because the structure becomes thermodynamically more stable as force acting on each atom becomes closer to zero.

The search system 100 (search method, or program) according to Embodiment 1 can include not only an aspect in which a stable structure such as the one described above is searched for and output to a user, but also an aspect in which data necessary for a user to search for a stable structure such as the one described above is output. That is, a process of searching for a stable structure need not necessarily be completed in the search system 100 (search method, or program).

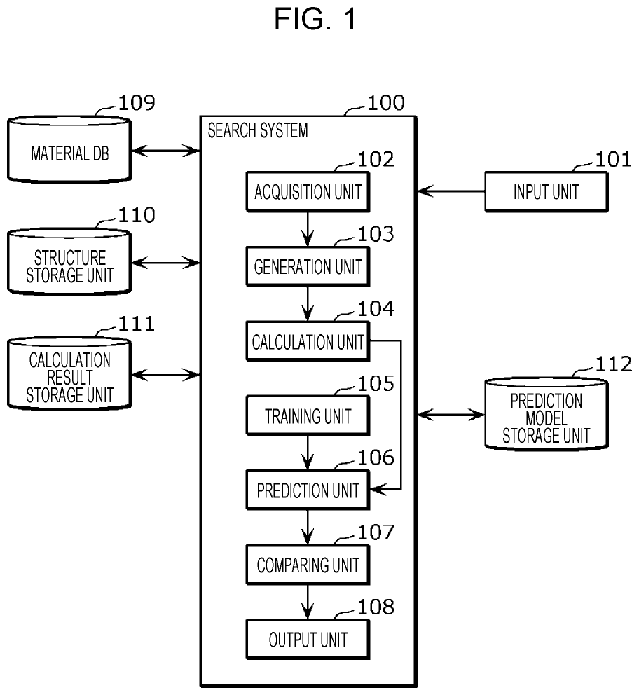

FIG. 1 is a block diagram illustrating an overall configuration including the search system 100 according to Embodiment 1. The search system 100 is, for example, a computer such as a personal computer or a server. As illustrated in FIG. 1, the search system 100 includes an acquisition unit 102, a generation unit 103, a calculation unit 104, a training unit 105, a prediction unit 106, a comparing unit 107, and an output unit 108. As peripheral constituents of the search system 100, an input unit 101, a material database (DB) 109, a structure storage unit 110, a calculation result storage unit 111, and a prediction model storage unit 112 are provided. Note that the peripheral constituents of the search system 100 may be included in the constituent elements of the search system 100. The generation unit 103 and the training unit 105 of the search system 100 are also constituent elements of a prediction model construction device.

Details of the constituent elements illustrated in FIG. 1 are described below.

### Input Unit 101

The input unit 101 is an input interface that receives user's input, and acquires information concerning a composition of a search target material by user's input and outputs the information to the acquisition unit 102. The information concerning the composition is, for example, composition formula information expressed in a character string format. The composition formula information can be, for example, expressed as “Li₁₂Mn₆Ni₆O₂₄”. This indicates that the composition of the search target material is made up of 12 lithium (Li) atoms, 6 manganese (Mn) atoms, 6 nickel (Ni) atoms, and 24 oxygen (O) atoms. The input unit 101 is, for example, configured to include a keyboard, a touch sensor, a touch pad, a mouse, or the like.

### Acquisition Unit 102

The acquisition unit 102 acquires composition formula information from the input unit 101 and acquires, from the material database 109, an atomic arrangement structure of a known material similar to a composition of a search target material included in the composition formula information. The “similar” as used herein means, for example, that elements included in the composition of the search target material and elements included in a composition of the known material differ only partially. The “similar” means that the composition of the known material includes at least one of the elements included in the composition of the search target material. The “similar” means that the composition of the search target material can be obtained by expanding processing or substituting processing of an atomic arrangement structure of the known material.

The material database 109 stores therein in advance known material data including a composition, a structure, and the like of each of one or more materials. The material database 109 is, for example, a storage medium such as a hard disk drive or a non-volatile semiconductor memory. Note that the material database 109 may be, for example, a public database such as Materials Project. The structure storage unit 110, the calculation result storage unit 111, and the prediction model storage unit 112, which will be described later, have similar configurations. The known material data includes, for example, information described in a crystallographic information common data format (Crystallographic Information File (CIF)). However, a format in which the information is described is not limited to a CIF data format and may be any format, such as a composition formula, a crystal structure, or a lattice vector, by which computation of structure optimization can be performed by ab initio calculation or the like.

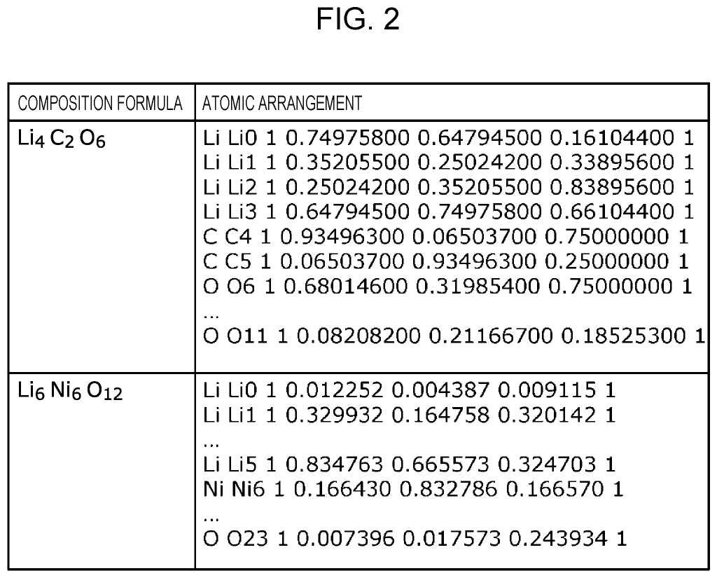

FIG. 2 illustrates an example of data stored in the material database 109 according to Embodiment 1. The material database 109 of the present disclosure stores therein data described in a format called a CIF. In the CIF, a composition formula indicative of a composition of a material, a length of a unit lattice vector, an angle at which atoms cross each other, atomic arrangement in a unit lattice, and the like are described. In the example illustrated in FIG. 2, atomic arrangement information concerning a material “Li₄C₂O₆” and a material “Li₆Ni₆O₁₂” is illustrated. In FIG. 2, the left column illustrates a composition formula indicative of a composition of a material, and the right column illustrates atomic arrangement concerning the composition of the material. In the atomic arrangement, atomic coordinates (an x coordinate, a y coordinate, and a z coordinate) of each atom (for example, in the case of a Li atom, four atoms in total “Li0” to “Li3”) or the like is described. Note that numerals such as “0” in “Li0” are merely given to distinguish elements of the same kind.

The acquisition unit 102 outputs composition formula information acquired from the input unit 101 and known material data acquired from the material database 109 to the generation unit 103.

### Generation Unit 103

The generation unit 103 performs expanding processing and substituting processing on an atomic arrangement structure (known structure) of a known material acquired from the acquisition unit 102. The generation unit 103 thus generates initial structures representing a composition of a search target material included in composition formula information acquired from the acquisition unit 102. That is, the generation unit 103 (a first step) acquires initial structures, which are atomic arrangement structures in a three-dimensional space which the composition of the material can take. The “initial structure” as used herein is any structure that has the same composition as the search target material and is any structure of atomic arrangement concerning the composition of the search target material. That is, at least a part of the initial structure is different from a stable structure. Furthermore, the generation unit 103 (the first step) acquires a known structure that is an atomic arrangement structure of a known material similar to the composition of the material in a three-dimensional space and generates initial structures on the basis of the known structure.

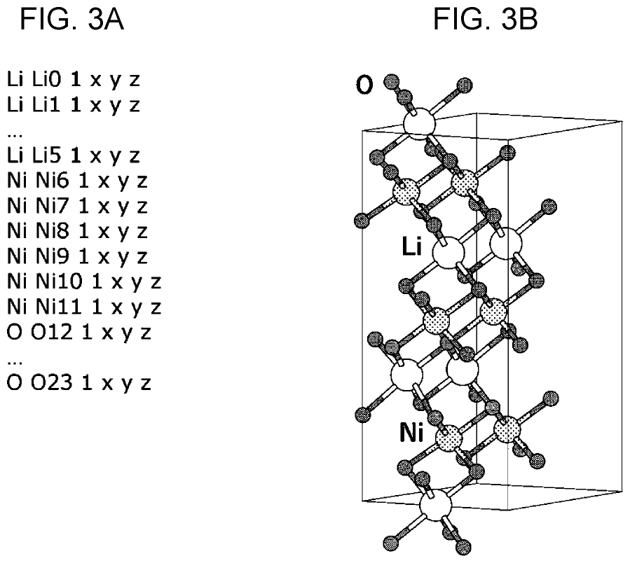

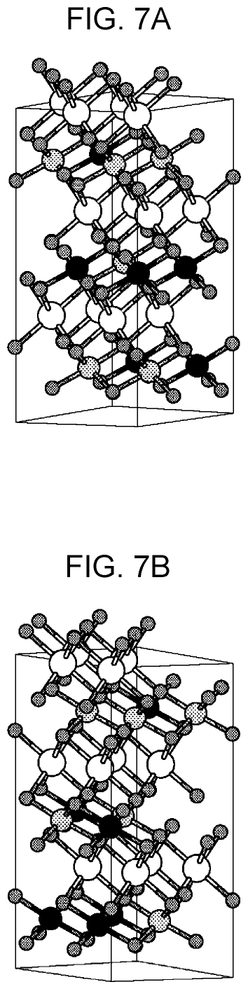

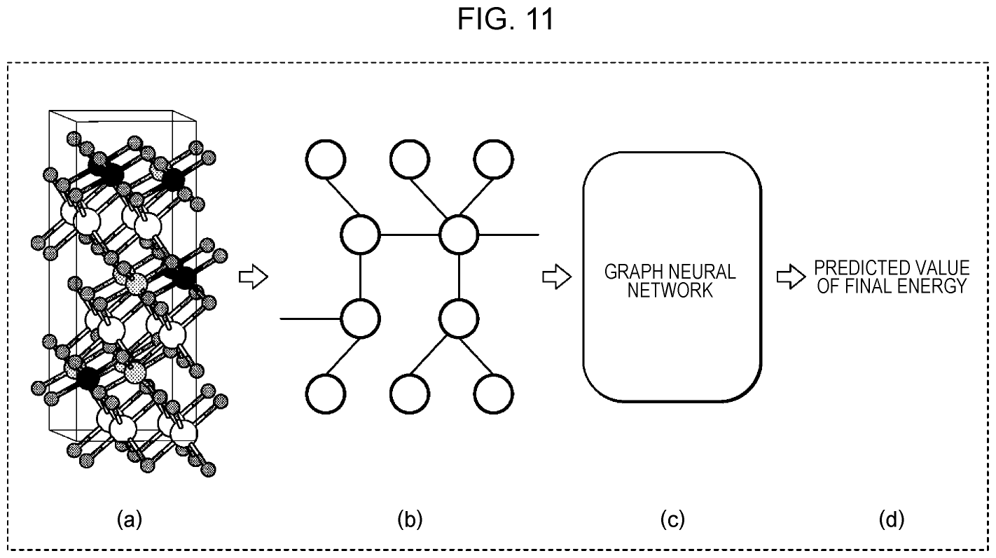

FIGS. 3A and 3B to FIGS. 5A and 5B illustrate an example of a process for generating an initial structure by the generation unit 103 according to Embodiment 1. FIGS. 3A, 4A, and 5A each illustrate a CIF of an initial structure, and FIGS. 3B, 4B, and 5B each illustrate a unit lattice, i.e., atomic arrangement of a crystal structure indicated by the CIF of the initial structure. As an example, it is assumed here that a composition formula of the known material is Li₆Ni₆O₁₂ and a composition formula of the search target material is Li₁₂Mn₆Ni₆O₂₄. In FIGS. 3B, 4B, and 5B, the smallest sphere represents an O atom, the sphere without hatching represents a Li atom, the sphere with hatching that has a similar size to the Li atom represents a Ni atom, and the black sphere represents a Mn atom. The same applies to FIG. 7A, FIG. 7B, and FIG. 11, which will be described later.

FIGS. 3A and 3B illustrate a CIF and atomic arrangement of the known structure. The generation unit 103 first generates an arrangement structure of Li₁₂Ni₁₂O₂₄ by expanding the known structure. The “expanding” as used herein refers to copying a structure to be expanded (in this example, the known structure) repeatedly in at least one of three-dimensional directions (the x direction, the y direction, and the z direction). That is, the generation unit 103 (the first step) includes a process of expanding the known structure in at least a one-dimensional direction.

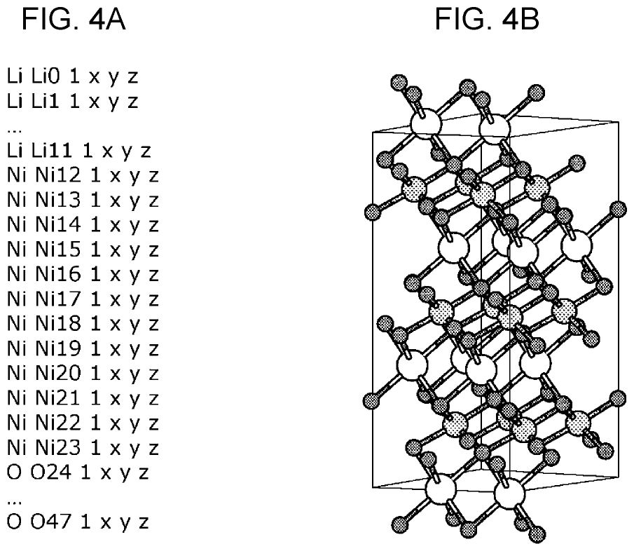

FIGS. 4A and 4B illustrate a CIF and atomic arrangement of the structure obtained by expanding the known structure. The structure illustrated in FIG. 4B is obtained by repeating the structure illustrated in FIG. 3B two times. As illustrated in FIGS. 4A and 4B, the number of atoms is made identical to the number of atoms included in the composition of the search target material by expanding the known structure. For example, the number of Li atoms in the known structure is 6 in total (“Li0” to “Li5”), whereas the number of Li atoms in the expanded structure is 12 in total (“Li0” to “Li11”), which is identical to the number of Li atoms in the composition of the search target material.

Next, the generation unit 103 substitutes 6 Ni atoms among 12 Ni atoms of the expanded structure (Li₁₂Ni₁₂O₂₄) with Mn atoms. That is, the composition of the known material contains at least one kind of element different from the elements contained in the composition of the search target material. The generation unit 103 (an acquisition step) includes a process of substituting the different kind of element with a same kind of element as an element contained in the composition of the search target material.

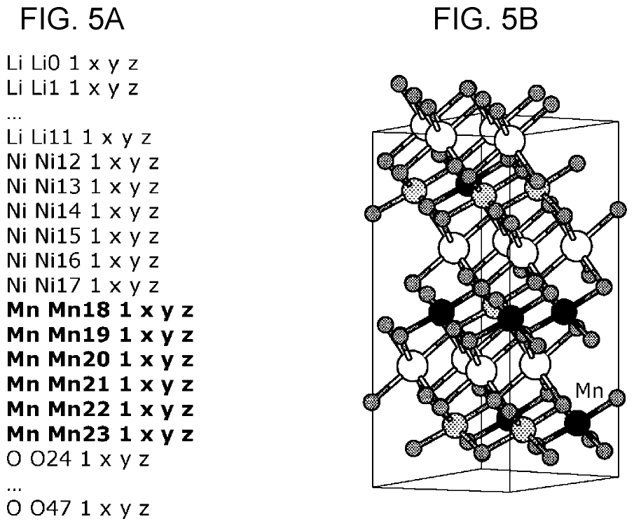

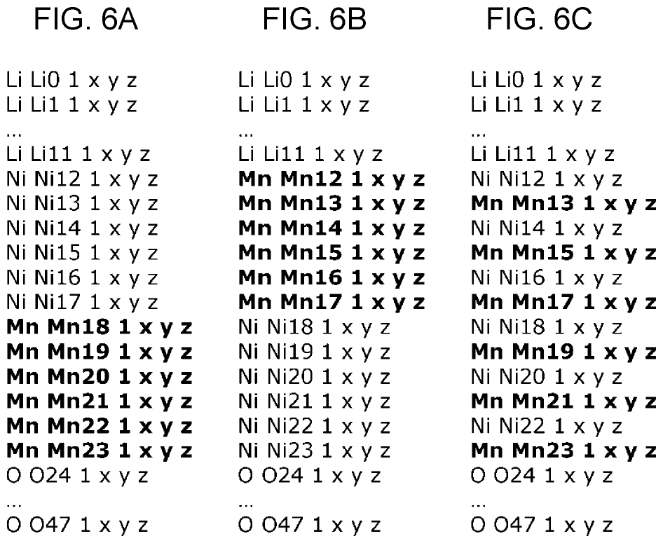

FIGS. 5A and 5B illustrate a CIF and atomic arrangement of the structure obtained after the substitution. In FIGS. 5A and 5B, 6 Ni atoms “Ni18” to “Ni23” among the 12 Ni atoms in the expanded structure have been substituted with 6 Mn atoms “Mn18” to “Mn23”. As illustrated in FIGS. 5A and 5B, elements contained in the structure obtained after the substitution are made identical to the elements contained in the composition of the search target material by substituting an element contained in the expanded structure.

As illustrated in FIGS. 6A to 6C, 7A, and 7B, plural combinations are conceivable as the structure after the substitution depending on which Ni atoms are substituted with Mn atoms. FIGS. 6A to 6C illustrate an example of initial structures generated by the generation unit 103 according to Embodiment 1. FIG. 7A illustrates an example of steric arrangement of an initial structure generated by the generation unit 103 according to Embodiment 1, and FIG. 7B illustrates another example of steric arrangement of an initial structure generated by the generation unit 103 according to Embodiment 1.

FIG. 6A illustrates a structure obtained in a case where “Ni18” to “Ni23” have been substituted with “Mn18” to “Mn23”. FIG. 6B illustrates a structure obtained in a case where “Ni12” to “Ni17” have been substituted with “Mn12” to “Mn17”. FIG. 6C illustrates a structure obtained in a case where “Ni13”, “Ni15”, “Ni17”, “Ni19”, “Ni21”, and “Ni23” have been substituted with “Mn13”, “Mn15”, “Mn17”, “Mn19”, “Mn21”, and “Mn23”. In the embodiment, the number of ways of selecting 6 Ni atoms to be substituted with Mn atoms from among 12 Ni atoms which Li₁₂Mn₆Ni₆O₂₄ can take is ₁₂C₆=924, that is, the generation unit 103 generates 924 initial structures.

The generation unit 103 outputs the generated initial structures to the structure storage unit 110. Note that the generation unit 103 may output all of the generated initial structures to the structure storage unit 110 or may output only initial structures selected by performing screening as to similar structures from a perspective of symmetry by using a known program or the like.

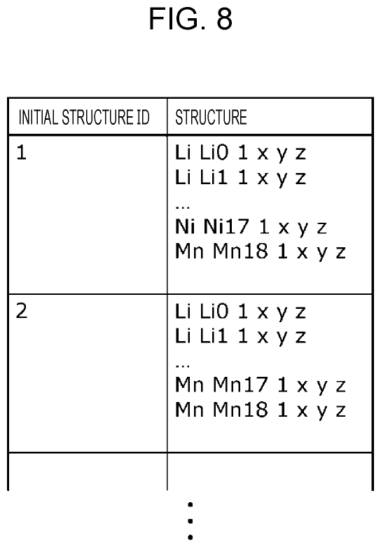

The structure storage unit 110 stores therein the initial structures generated by the generation unit 103. Data of each of the initial structures is stored in a description format, such as a composition formula, a crystal structure, and a lattice vector, by which computation of structure optimization can be performed by ab initio calculation or the like, as in the material database 109. FIG. 8 illustrates an example of data stored in the structure storage unit 110 according to Embodiment 1. In FIG. 8, the left column illustrates initial structure identifiers (IDs) allocated to distinguish the initial structures, and the right column illustrates atomic arrangement of the initial structures.

### Calculation Unit 104

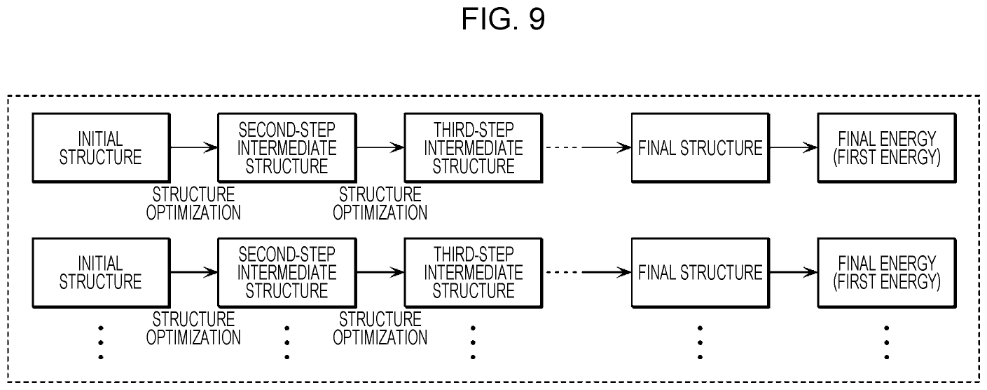

As illustrated in FIG. 9, the calculation unit 104 acquires one or some of the initial structures from the structure storage unit 110 and performs structure optimization on the acquired initial structures. The calculation unit 104 performs processing of calculating energy (first energy) corresponding to final structures obtained by repeating structure optimization. FIG. 9 illustrates an example of a process of calculating first energy by the calculation unit 104 according to Embodiment 1.

That is, the calculation unit 104 (a second step) performs structure optimization on one or some of the initial structures and calculates first energy corresponding to structurally-optimized atomic arrangement structures. The “first energy” as used herein sometimes refers to energy corresponding to a final structure obtained by repeating structure optimization, and sometimes refers to energy corresponding to an intermediate structure that has not reached to a final structure yet. In the embodiment, the calculation unit 104 performs processing of performing structure optimization and calculating first energy corresponding to final structures by using an ab initio calculation package such as Vienna Ab initio Simulation Package (VASP). The “energy” in the present disclosure may mean “potential energy”.

The “final structure” as used herein refers to a structure obtained by performing structure optimization on an initial structure and is such a structure that force acting on each atom included in the structure is equal to or less than a threshold value. The “intermediate structure” is a structure obtained by performing structure optimization on an initial structure and is such a structure that force acting on at least one atom included in the structure is larger than the threshold value, that is, a structure that has not reached the final structure yet.

In the structure optimization, the calculation unit 104 calculates force F acting on each atom included in a structure to be processed and searches for a structure (i.e., a final structure) in which the calculated force F acting on each atom is equal to or less than a threshold value. The threshold value may be a value close to zero, as described earlier. Specifically, in a case where force F acting on at least one atom is larger than the threshold value in a structure obtained by performing structure optimization, the calculation unit 104 adjusts a position of each atom so that the force F becomes small by moving each atom in a direction in which the force F is applied. The calculation unit 104 repeats processing of calculating the force F on each atom and processing for adjusting a position of each atom, which are regarded as one structure optimization, and finishes the structure optimization in a case where a structure (i.e., a final structure) in which the force F on all atoms is equal to or less than the threshold value is obtained. Then, the calculation unit 104 calculates energy corresponding to the obtained final structure, that is, final energy.

In ab initio calculation based on Density Functional Theory (DFT), it takes, for example, several tens of seconds to several minutes to calculate the force F acting on each atom. It is necessary to perform processing of adjusting a position of each atom, for example, approximately several times to several tens of times until an initial structure reaches a final structure. Therefore, the calculation unit 104 needs to repeat, for each initial structure, structure optimization, which takes several tens of seconds to several minutes, approximately several times to several tens of times in order to obtain a final structure from an initial structure, and therefore it takes approximately several tens of minutes to several hours in total.

The calculation unit 104 outputs an initial structure, a final structure obtained by repeatedly performing structure optimization on the initial structure, and final energy corresponding to the calculated final structure to the calculation result storage unit 111.

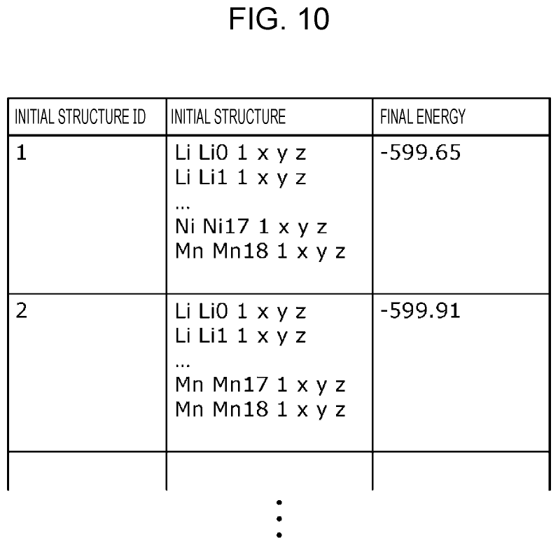

The calculation result storage unit 111 stores therein a combination of final energy calculated by the calculation unit 104 and a corresponding initial structure. FIG. 10 illustrates an example of data stored in the calculation result storage unit 111 according to Embodiment 1. In FIG. 10, the left column illustrates an initial structure ID, the middle column illustrates atomic arrangement of an initial structure, and the right column illustrates final energy corresponding to a final structure obtained by performing structure optimization on the initial structure. As described above, the calculation result storage unit 111 need just store at least a combination of an initial structure and final energy of a final structure. In Embodiment 1, the calculation result storage unit 111 further stores therein atomic arrangement of a final structure.

### Training Unit 105

The training unit 105 acquires an initial structure and final energy of a final structure from the calculation result storage unit 111, and trains a prediction model by using the acquired initial structure and final energy. As for a combination of an input and an output which the prediction model learns, for example, the input is an initial structure, and the output is final energy.

That is, the training unit 105 (a seventh step) trains the prediction model by machine learning to output energy corresponding to a structure (in this example, a final structure) obtained by performing structure optimization on any atomic arrangement structure (in this example, an initial structure) in response to input of the atomic arrangement structure by using a learning dataset. The learning dataset includes an initial structure as input data and includes, as correct answer data, energy corresponding to an atomic arrangement structure (in this example, a final structure) obtained by performing structure optimization on the initial structure.

In the embodiment, the prediction model is a graph neural network using a graph structure as an input. The graph neural network is, for example, a Crystal Graph Convolutional Neural Network (CGCNN) or a Material Graph Network (MEGNet). In the embodiment, the prediction model is the MEGNet. The MEGNet is a graph neural network that uses, as feature amounts, not only nodes (node points, vertexes) and edges (branches, links), but also global state amounts representing features of a whole target system.

FIG. 11 illustrates an example of a process of performing machine learning of a prediction model by the training unit 105 according to Embodiment 1. The training unit 105 first converts atomic coordinates and kinds of atoms of an initial structure such as the ones illustrated in FIG. 11(a) into a graph structure such as the one illustrated in FIG. 11(b). In the graph structure, a node corresponds to an atom of the initial structure, and an edge corresponds to a bond between atoms of the initial structure. Next, the training unit 105 inputs the graph structure obtained by the conversion to a graph neural network such as the one illustrated in FIG. 11(c). Next, the training unit 105 compares a predicted value of final energy illustrated in FIG. 11(d) output from the graph neural network and final energy as correct answer data. Then, the training unit 105 updates a weight of the graph neural network in a case where the predicted value of the final energy output from the graph neural network is deviated from the final energy as the correct answer data. In this way, the training unit 105 performs machine learning of the prediction model by supervised learning by using learning datasets.

The training unit 105 outputs the prediction model that has completed machine learning, that is, the trained model to the prediction unit 106 and the prediction model storage unit 112. The prediction model that has completed machine learning has been trained by machine learning to output, as second energy, which will be described later, energy corresponding to a structure (in this example, a final structure) obtained by performing structure optimization on any atomic arrangement structure (in this example, an initial structure) in response to input of the atomic arrangement structure. This prediction model is a model that has been trained by machine learning using a first learning dataset including an initial structure as input data and including first energy (in this example, final energy) corresponding to the initial structure as correct answer data.

The prediction model storage unit 112 stores therein a structure and a weight of the graph neural network regarding the prediction model that has been trained by machine learning by the training unit 105.

### Prediction Unit 106

The prediction unit 106 acquires initial structure(s) for which final energy has not been calculated yet from the structure storage unit 110. Then, the prediction unit 106 predicts final energy of the initial structure(s) by inputting the initial structure(s) to the prediction model acquired from the training unit 105, that is, the trained prediction model.

The “initial structure(s) for which final energy has not been calculated yet” refer to structure(s) that are not the one or some initial structures for which energy has been calculated by the calculation unit 104 among the initial structures and refer to other initial structure(s). That is, the prediction unit 106 (a third step) predicts second energy corresponding to atomic arrangement structures obtained in a case where structure optimization is performed on the other initial structure(s) among the initial structures by using a prediction model for the other initial structure(s). In this example, the second energy is predicted values of final energy corresponding to final structures obtained in a case where structure optimization is performed on the other initial structure(s).

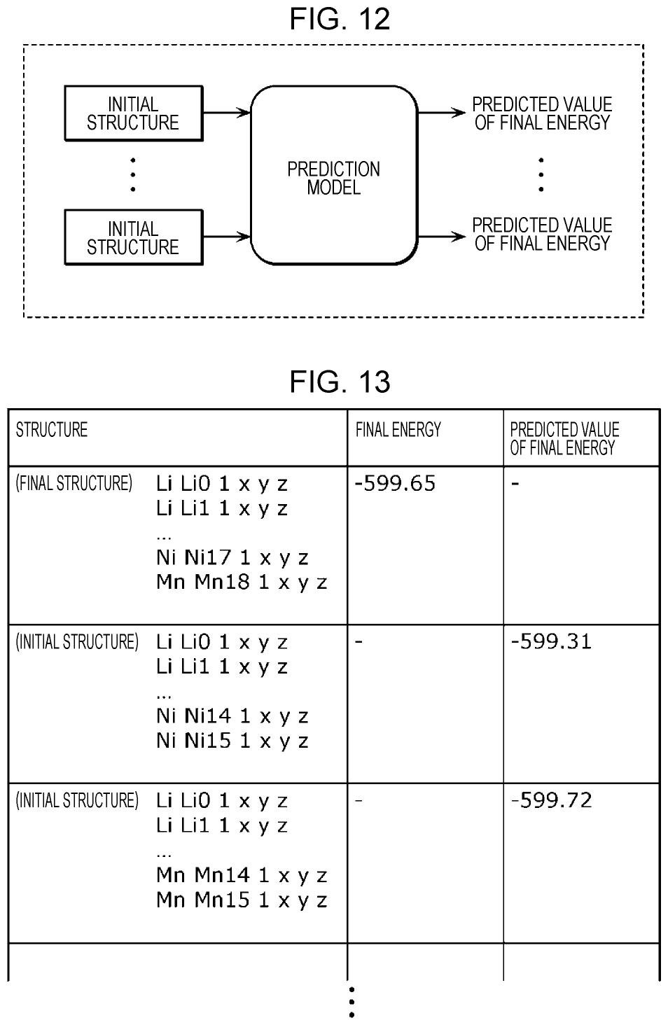

FIG. 12 illustrates an example of a process of predicting second energy by the prediction unit 106 according to Embodiment 1. The prediction unit 106 converts the initial structures into graph structures and inputs the converted initial structures to the prediction model. In FIG. 12, illustration of a process of converting the initial structures into the graph structures is omitted. In this way, the prediction model outputs predicted values of final energy, that is, second energy corresponding to final structures obtained in a case where structure optimization is performed on the input initial structures.

That is, although a prediction model such as the one disclosed in Non Patent Literature 2 outputs predicted values of energy corresponding to input initial structures, the prediction model according to Embodiment 1 outputs predicted values of energy corresponding to structures obtained in a case where structure optimization is performed on the input initial structures, that is, intermediate structures or final structures. The predicted values of energy output by the prediction model correspond to energy corresponding to structures obtained by actually performing structure optimization on the initial structures by the calculation unit 104 although the predicted values depend on prediction accuracy of the prediction model.

Therefore, in Embodiment 1, energy corresponding to structurally optimized structures (e.g., intermediate structures or final structures) can be acquired by using the prediction model without performing structure optimization on the initial structures several to several tens of times. Therefore, in Embodiment 1, computation related to structure optimization can be omitted to some extent, and therefore a computation cost can be reduced.

The prediction unit 106 outputs initial structures and predicted values of final energy corresponding to the initial structures to the comparing unit 107.

### Comparing Unit 107

The comparing unit 107 acquires combinations of initial structures and predicted values of final energy from the prediction unit 106. The comparing unit 107 acquires combinations of final structures and final energy from the calculation result storage unit 111. Then, the comparing unit 107 generates a list in which the combinations of initial structures and predicted values of final energy and the combinations of final structures and final energy are arranged.

FIG. 13 illustrates an example of data generated by the comparing unit 107 according to Embodiment 1. In FIG. 13, the left column illustrates atomic arrangement of an initial structure or a final structure, the middle column illustrates final energy corresponding to the final structure, and the right column illustrates a predicted value of final energy corresponding to the initial structure. The comparing unit 107 sorts the final energy and the predicted values of the final energy in a predetermined order on the basis of the list. In Embodiment 1, the comparing unit 107 sorts the final energy and the predicted values of the final energy in an ascending order of energy values. Such sorting of the final energy and the predicted values of the final energy corresponds to processing of extracting a smallest value, in other words, a minimum value from among the final energy and the predicted values of the final energy.

That is, the comparing unit 107 (a fourth step) extracts third energy indicative of a minimum value on the basis of the first energy and the second energy. The first energy is final energy acquired from the calculation result storage unit 111, and the second energy is predicted values of final energy acquired from the prediction unit 106. In this example, the minimum value is a smallest value among the first energy and the second energy. That is, the third energy is a smallest value among the first energy and the second energy.

The comparing unit 107 outputs a list in which the final energy and the predicted values of the final energy are sorted as described above to the output unit 108.

### Output Unit 108

The output unit 108 displays, on a display, the initial structures and the predicted values of the final energy and the final structures and the final energy included in the list output by the comparing unit 107 in accordance with the predetermined order, that is, starting from a structure of smallest energy. That is, the output unit 108 (a fifth step) outputs the third energy, a first structure that is an atomic arrangement structure corresponding to the third energy, or the third energy and the first structure.

Note that the output unit 108 may display, on the display, only the third energy and an atomic arrangement structure corresponding to the third energy. The output unit 108 may display, on the display, the list in which the final energy and the predicted values of the final energy have not been sorted by the comparing unit 107. That is, the output unit 108 (a sixth step) may output the first energy and the second energy. In this case, the extracting processing (fourth step) performed by the comparing unit 107 is unnecessary.

### Embodiment 1: Verification of Prediction Accuracy

Verification of prediction accuracy of the prediction unit 106 according to Embodiment 1 is described below. This verification is intended to check whether or not the prediction unit 106 can predict a stable structure for each of substances having 21 kinds of compositions constituted by 48 atoms including a Li atom and a Mn atom and further including at least one of a Ni atom and an O atom.

First, in the verification, 1086 combinations of initial structures and final energy in total were prepared for the substances having 21 kinds of compositions. That is, final structures were obtained by performing structure optimization on each of the 1086 initial structures in total, and final energy corresponding to the obtained final structures was calculated. Then, among the 1086 combinations, 328 combinations, which account for 30% of all combinations, were used as data for verification (test data), and 758 combinations, which account for remaining 70%, were used as data for learning (train data).

Machine learning of the prediction model was performed while using, as the data for learning, a learning dataset including initial structures as input data and final energy as correct answer data. Final energy of the data for verification was predicted by using the prediction model trained by machine learning. That is, by inputting the initial structures included in the data for verification to the prediction model trained by machine learning, predicted values of final energy corresponding to the initial structures output from the prediction model were acquired.

As an evaluation index of prediction accuracy, in which place an atomic arrangement structure considered as being most stable among final structures obtained by actually performing structure optimization on the initial structures was ranked was predicted by the prediction model. In this way, it is possible to evaluate whether or not screening using the prediction model is possible.

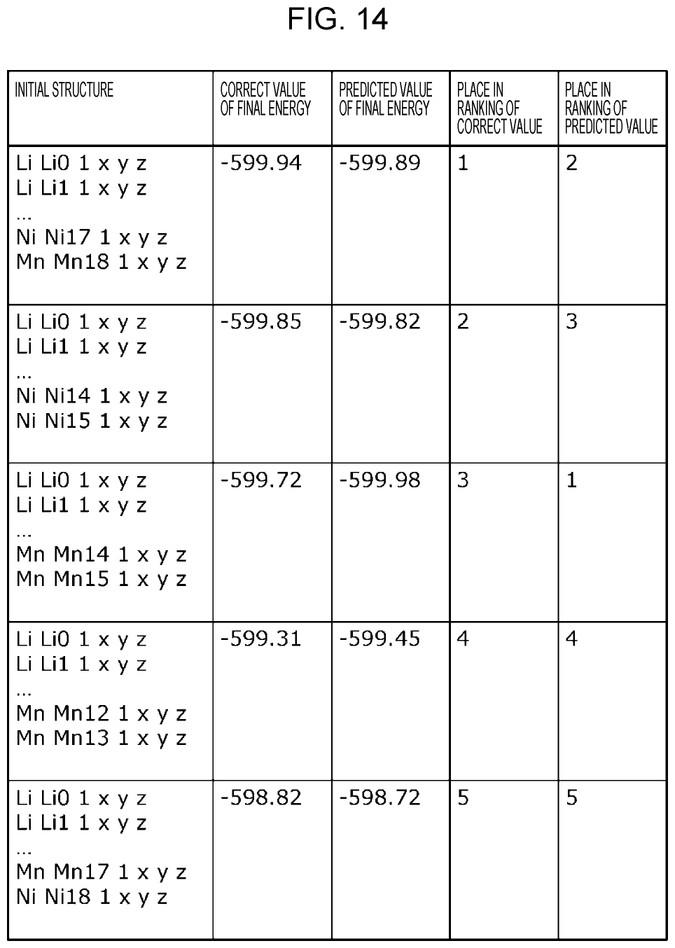

FIG. 14 illustrates an example of evaluation of prediction accuracy of the prediction unit 106 according to Embodiment 1. In FIG. 14, an initial structure, a correct value of final energy corresponding to the initial structure, a predicted value of the final energy corresponding to the initial structure, a place in ranking of the correct value, and a place in ranking of the predicted value are illustrated in this order from the leftmost column. The “correct value of the final energy” is final energy corresponding to a final structure obtained by actually performing structure optimization on the initial structure. The “predicted value of the final energy” is a predicted value of final energy output from the prediction model by inputting the initial structure to the prediction model. The “place in ranking” is decided assuming that a final structure of a smallest correct value of final energy or a smallest predicted value of final energy is in the first place in ranking.

In the example illustrated in FIG. 14, it was predicted by the prediction unit 106 that an atomic arrangement structure considered as being most stable among structures obtained by actually performing structure optimization is the second most stable atomic arrangement structure.

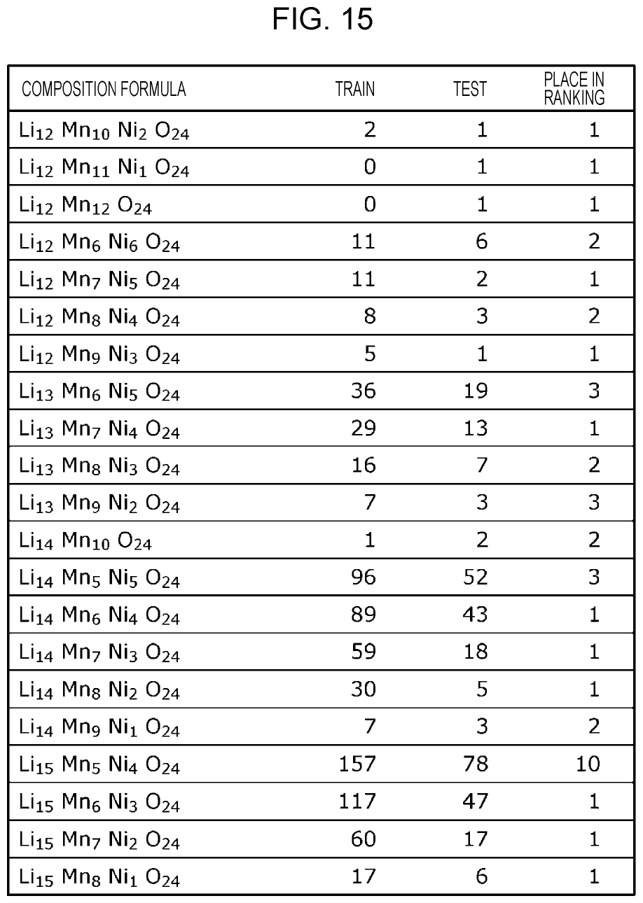

FIG. 15 illustrates a result of verification of prediction accuracy of the prediction unit 106 according to Embodiment 1. In FIG. 15, a composition formula of a substance, the number of pieces of data for learning concerning the substance, the number of pieces of data for verification concerning the substance, and a place in ranking are illustrated in this order from the leftmost column. The “place in ranking” indicates a place in ranking of stability predicted by the prediction unit 106 as for an atomic arrangement structure considered as being actually most stable among the data for verification concerning the substance.

It is concerned here that as the number of pieces of data for verification increases, prediction accuracy of the prediction unit 106 decreases. However, for example, as for Li₁₄Mn₅Ni₅O₂₄, it was predicted by the prediction unit 106 that an atomic arrangement structure considered as being actually most stable among 52 pieces of data for verification is the third most stable structure. Furthermore, for example, as for Li₁₅Mn₅Ni₄O₂₄, it was predicted by the prediction unit 106 that an atomic arrangement structure considered as being actually most stable among 78 pieces of data for verification is the tenth most stable structure.

As described above, these results show that the prediction unit 106 can predict, for any substance having any composition, that an atomic arrangement structure considered as being actually most stable is a structure ranked in terms of stability within 20% of all pieces of data for verification concerning the substance. That is, the results show that even when the number of pieces of data for verification increases, prediction accuracy of the prediction unit 106 hardly decreases. Here, the prediction unit 106 may be able to predict, for any substance having any composition, that an atomic arrangement structure considered as being actually most stable is a structure ranked in terms of stability within 17% of all pieces of data for verification concerning the substance, moreover within 13% of all pieces of data for verification concerning the substance.

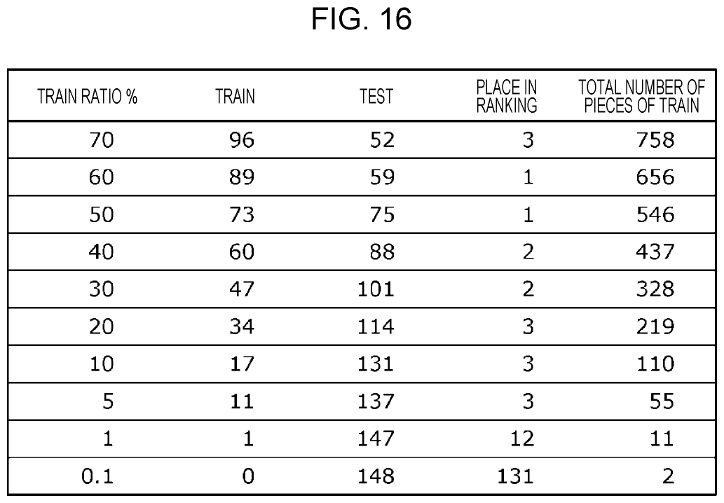

FIG. 16 illustrates a result of verification of a correlation between prediction accuracy of the prediction unit 106 and a ratio of data for learning according to Embodiment 1. Specifically, FIG. 16 illustrates a result obtained in a case where prediction accuracy of the prediction unit 106 was verified while changing a ratio of data for learning as for a substance having a composition Li₁₄Mn₅Ni₅O₂₄. The “ratio of data for learning” is a ratio of the number of pieces of data for learning to a sum of the number of pieces of data for learning and the number of pieces of data for verification concerning the substance having a composition Li₁₄Mn₅Ni₅O₂₄ and is expressed in percentage. In FIG. 16, a ratio of data for learning concerning the substance, the number of pieces of data for learning concerning the substance, the number of pieces of data for verification concerning the substance, a place in ranking, and a total number of pieces of data for learning concerning all substances are illustrated in this order from the leftmost column. The “place in ranking” indicates a place in ranking of stability predicted by the prediction unit 106 concerning an atomic arrangement structure considered as being actually most stable among the data for verification concerning the substance.

As illustrated in FIG. 16, a decrease in prediction accuracy of the prediction unit 106 was hardly observed even when the ratio of the data for learning was decreased. Even when the ratio of the data for learning was 5%, it was predicted by the prediction unit 106 that an atomic arrangement structure considered as being actually most stable among the 137 pieces of data for verification was the third most stable structure.

### Embodiment 1: Description of Operation

Next, operation of the search system 100 is described.

### Flowchart

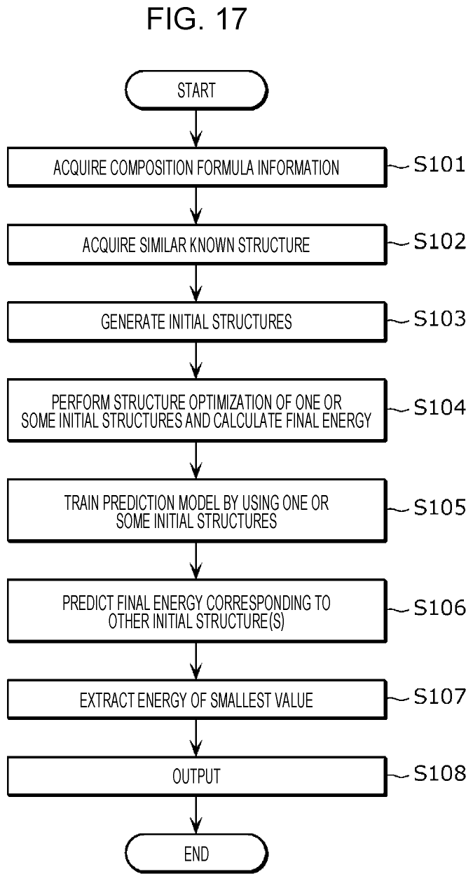

FIG. 17 is a flowchart illustrating an operation example of the search system 100 according to Embodiment 1.

### Step S101

The input unit 101 acquires composition formula information by user's input, and outputs the acquired composition formula information to the acquisition unit 102.

### Step S102

The acquisition unit 102 acquires an atomic arrangement structure of a known material similar to a composition of a search target material included in the composition formula information from the material database 109 and outputs the acquired similar known material to the generation unit 103.

### Step S103

The generation unit 103 performs expanding processing and substituting processing on the atomic arrangement structure of the known structure acquired in step S102. The generation unit 103 thus generates initial structures representing the composition of the search target material included in the composition formula information and outputs the initial structures to the structure storage unit 110.

### Step S104

The calculation unit 104 performs structure optimization on one or some initial structures among the initial structures generated in step S103 and calculates final energy corresponding to final structures obtained by performing the structure optimization. Then, the calculation unit 104 outputs a calculation result to the calculation result storage unit 111. Here, in a case where the generation unit 103 (the first step) acquires n (n is an integer of two or more) initial structures, the one or some initial structures in the calculation unit 104 (the second step) are m (m is an integer that is equal to or more than one and is less than n) initial structures. Here, “m” is 90% or less of “n”. “m” may be 1% or more and 90% or less of “n”. That is, the number of one or some initial structures in the calculation unit 104 (the second step) is 90% or less of the total number of initial structures.

### Step S105

The training unit 105 performs machine learning of a prediction model that is a graph neural network by using, as a learning dataset, combinations of the final energy calculated in step S104 and initial structures. Then, the training unit 105 outputs the prediction model trained by the machine learning to the prediction unit 106 and the prediction model storage unit 112. Here, the number of learning datasets is identical to the number of one or some initial structures and is m.

### Step S106

The prediction unit 106 acquires initial structure(s) for which final energy has not been calculated yet, that is, other initial structure(s) among the initial structures from the structure storage unit 110. Then, the prediction unit 106 calculates predicted values of final energy corresponding to the other initial structure(s) by the prediction model trained by the machine learning in step S105. Here, the number of other initial structures is a number obtained by subtracting the number of one or some initial structures from the total number of initial structures. That is, the other initial structure(s) in the prediction unit 106 (the third step) are (n-m) initial structures.

Note that although the prediction model is a prediction model trained by machine learning in step S105 in Embodiment 1, the prediction model may be another prediction model trained in advance acquired from the prediction model storage unit 112.

### Step S107

The comparing unit 107 generates a list in which the final energy calculated in step S104 and the predicted values of the final energy calculated in step S106 are sorted in an ascending order of energy values and outputs the generated list to the output unit 108. That is, the comparing unit 107 extracts energy of a smallest value from among the final energy and the predicted values of the final energy.

### Step S108

The output unit 108 outputs the initial structures and the predicted values of the final energy and the final structures and the final energy included in the list generated in step S107 by displaying them on a display in an ascending order of energy values.

As described above, in Embodiment 1, computation related to structure optimization is omitted by performing structure optimization only on one or some initial structures and using a prediction model for remaining other initial structure(s) instead of performing structure optimization on all initial structures. In Embodiment 1, it is therefore possible to search for an atomic arrangement structure considered as being thermodynamically most stable in a novel substance as in a case where structure optimization is performed on all initial structures and omit computation necessary for search to some extent. That is, in Embodiment 1, it is possible to reduce a computation cost and efficiently search for a stable atomic arrangement structure concerning a composition of a material as compared with a case where structure optimization is performed on all initial structures.

### Embodiment 2

A search system 200 (search method, or program) according to Embodiment 2 of the present disclosure is described in detail below with reference to the drawings. The search system 200 according to Embodiment 2 is different from the search system 100 according to Embodiment 1 in that not only initial structures, but also intermediate structures and final structures are used when machine learning of a prediction model is performed. Note that in the present embodiment, constituent elements identical to those in Embodiment 1 are given identical reference signs, and description thereof is omitted.

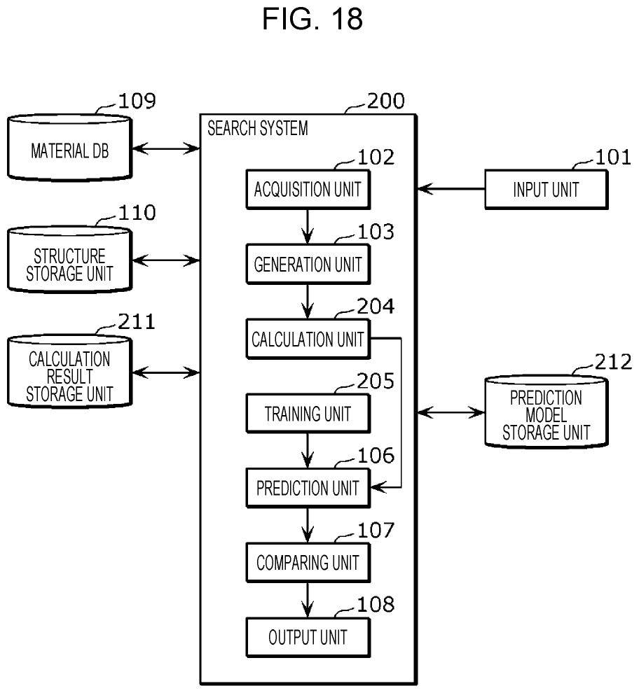

FIG. 18 is a block diagram illustrating an overall configuration including the search system 200 according to Embodiment 2. As illustrated in FIG. 18, the search system 200 includes an acquisition unit 102, a generation unit 103, a calculation unit 204, a training unit 205, a prediction unit 106, a comparing unit 107, and an output unit 108. As peripheral constituents of the search system 200, an input unit 101, a material database (DB) 109, a structure storage unit 110, a calculation result storage unit 211, and a prediction model storage unit 212 are provided. Note that the peripheral constituents of the search system 200 may be included in the constituent elements of the search system 200. The generation unit 103 and the training unit 205 of the search system 200 are also constituent elements of a prediction model construction device.

Details of each constituent element illustrated in FIG. 18 are described below. Note that constituent elements other than the calculation result storage unit 211, the prediction model storage unit 212, the calculation unit 204, and the training unit 205 are identical to those in Embodiment 1, and description thereof is omitted.

### Calculation Unit 204

The calculation unit 204 acquires one or some initial structures from the structure storage unit 110 and performs structure optimization on the acquired initial structures. The calculation unit 104 performs processing of calculating energy (first energy) corresponding to final structures obtained by repeating structure optimization.

The calculation unit 204 outputs the initial structures, final structures obtained by repeatedly performing structure optimization on the initial structures, and final energy corresponding to the calculated final structures to the calculation result storage unit 211. Then, in Embodiment 2, the calculation unit 204 also outputs intermediate structures obtained every time structure optimization is performed on the initial structures to the calculation result storage unit 211.

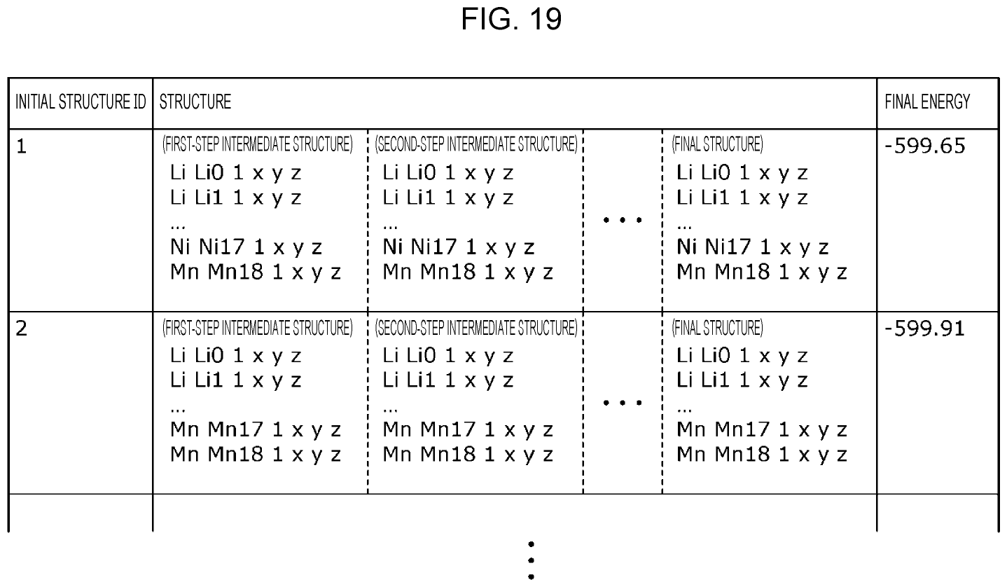

The calculation result storage unit 211 stores therein combinations of the final energy calculated by the calculation unit 204, corresponding initial structures, corresponding intermediate structures, and corresponding final structures. FIG. 19 illustrates an example of data stored in the calculation result storage unit 211 according to Embodiment 2. In FIG. 19, the left column illustrates an initial structure ID, the middle column illustrates atomic arrangement of an intermediate structure obtained every time structure optimization is performed and atomic arrangement of a final structure, and the right column illustrates final energy corresponding to the final structure. In FIG. 19, illustration of atomic arrangement of an initial structure is omitted.

### Training Unit 205

The training unit 205 acquires the initial structures, the intermediate structures, the final structures, and the final energy of the final structures from the calculation result storage unit 211 and trains a prediction model by using these.

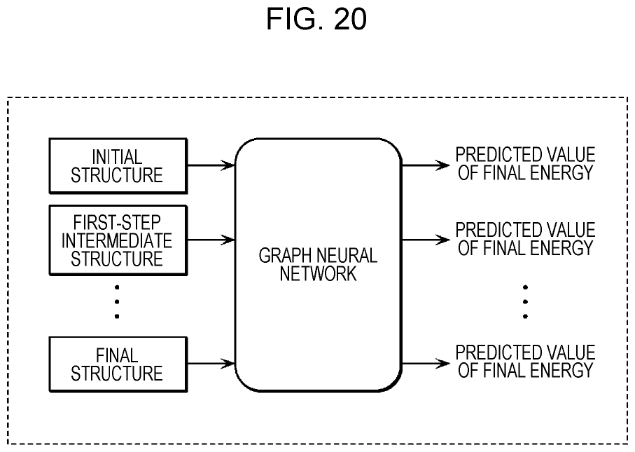

FIG. 20 illustrates an example of a process of performing machine learning of the prediction model by the training unit 205 according to Embodiment 2. As illustrated in FIG. 20, in Embodiment 2, input data included in a learning dataset includes not only an initial structure, but also an intermediate structure obtained every time structure optimization is performed and a final structure.

That is, in Embodiment 2, the training unit 205 performs machine learning of a prediction model by using not only a first learning dataset including an initial structure as input data and final energy as correct answer data, but also a second learning dataset including an intermediate structure or a final structure as input data and final energy as correct answer data. Therefore, in Embodiment 2, the prediction model is a model trained by machine learning by using not only the first learning dataset, but also the second learning dataset including a structurally optimized atomic arrangement structure, that is, an intermediate structure or a final structure as input data and including first energy corresponding to the structure, that is, final energy as correct answer data. Note that details of processing of the machine learning of the prediction model by the training unit 205 are similar to those in Embodiment 1, and therefore description thereof is omitted.

The training unit 205 outputs the prediction model that has completed the machine learning, that is, the trained model to the prediction unit 106 and the prediction model storage unit 212.

The prediction model storage unit 212 stores therein a structure and a weight of a graph neural network concerning the prediction model trained by machine learning by the training unit 205.

### Embodiment 2: Verification of Prediction Accuracy

Verification of prediction accuracy of the prediction unit 106 according to Embodiment 2 is described below. This verification is intended to check whether or not the prediction unit 106 can predict a stable structure as for each of substances having 21 kinds of compositions constituted by 48 atoms including a Li atom and a Mn atom and further including at least one of a Ni atom and an O atom, as in the verification in Embodiment 1.

Since contents of the verification are basically identical to those in Embodiment 1, description of the same contents is omitted. The verification of Embodiment 2 is different from the verification in Embodiment 2 in that a learning dataset used for machine learning of a prediction model includes not only the first learning dataset, but also the second learning dataset.

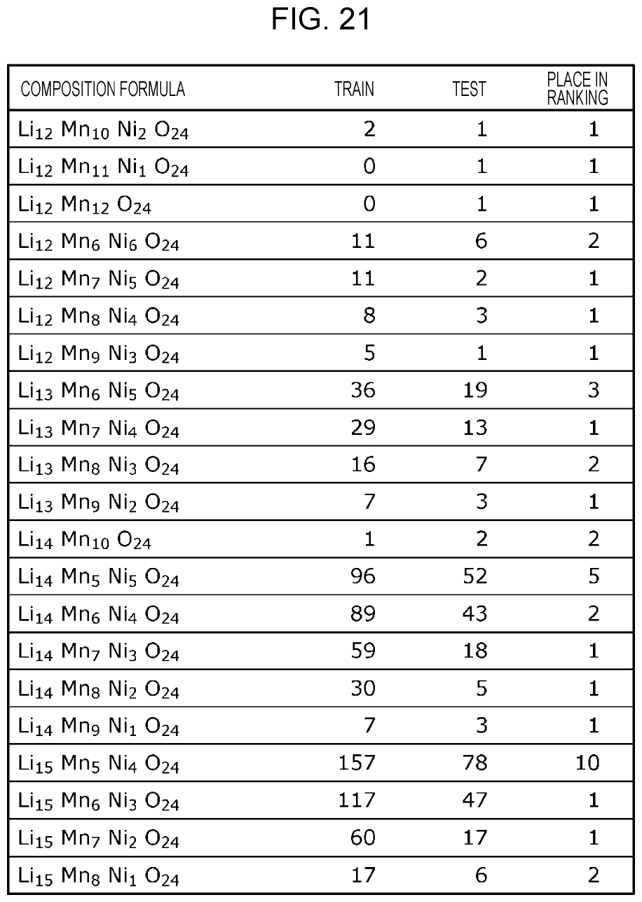

FIG. 21 illustrates a result of verification of prediction accuracy of the prediction unit 106 according to Embodiment 2. What is illustrated by each column in FIG. 21 is similar to that in FIG. 15 of Embodiment 1, and therefore description thereof is omitted.

It is concerned here that as the number of pieces of data for verification increases, prediction accuracy of the prediction unit 106 decreases. However, for example, as for Li₁₄Mn₅Ni₅O₂₄, it was predicted by the prediction unit 106 that an atomic arrangement structure considered as being actually most stable among 52 pieces of data for verification was the fifth most stable structure. Furthermore, for example, as for Li₁₅Mn₅Ni₄O₂₄, it was predicted by the prediction unit 106 that an atomic arrangement structure considered as being actually most stable among 78 pieces of data for verification was the tenth most stable structure.

As described above, these results show that the prediction unit 106 can predict, for any substance having any composition, that an atomic arrangement structure considered as being actually most stable is a structure ranked in terms of stability within 20% of all pieces of data for verification concerning the substance. That is, the results show that even when the number of pieces of data for verification increases, prediction accuracy of the prediction unit 106 hardly decreases. Here, the prediction unit 106 may be able to predict, for any substance having any composition, that an atomic arrangement structure considered as being actually most stable is a structure ranked in terms of stability within 17% of all pieces of data for verification concerning the substance, moreover within 13% of all pieces of data for verification concerning the substance.

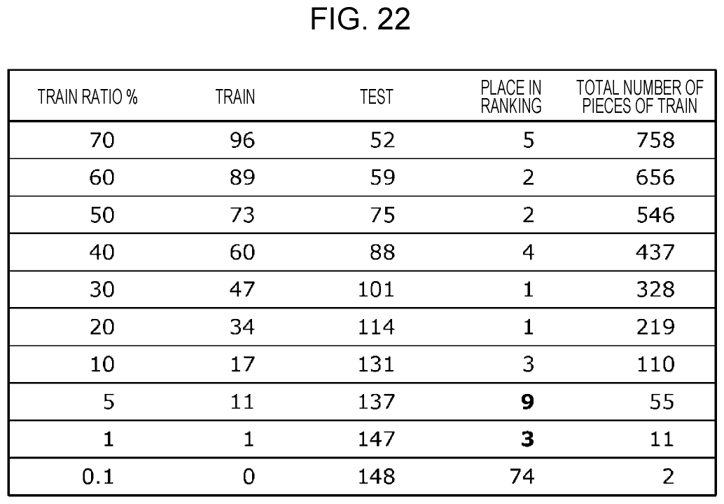

FIG. 22 illustrates a result of verification of a correlation between prediction accuracy of the prediction unit 106 and a ratio of data for learning according to Embodiment 2. Specifically, FIG. 22 illustrates a result obtained in a case where prediction accuracy of the prediction unit 106 was verified while changing a ratio of data for learning as for a substance having a composition Li₁₄Mn₅Ni₅O₂₄. What is illustrated by each column in FIG. 22 is similar to that in FIG. 16 of Embodiment 1, and therefore description thereof is omitted.

As illustrated in FIG. 22, a decrease in prediction accuracy of the prediction unit 106 was hardly observed even when the ratio of the data for learning was decreased. In Embodiment 2, even when the ratio of the data for learning was 1%, it was predicted by the prediction unit 106 that an atomic arrangement structure considered as being actually most stable among the 147 pieces of data for verification was the third most stable structure. On the other hand, in Embodiment 1, in a case where the ratio of the data for learning was 1%, it was predicted by the prediction unit 106 that an atomic arrangement structure considered as being actually most stable was the twelfth most stable structure. That is, in Embodiment 2, prediction may be performed with high accuracy even in a case where the ratio of the data for learning is low by performing machine learning of a prediction model by further using a learning dataset including a structurally optimized atomic arrangement structure, that is, an intermediate structure or a final structure as input data.

### Embodiment 2: Description of Operation

Next, operation of the search system 200 is described.

### Flowchart

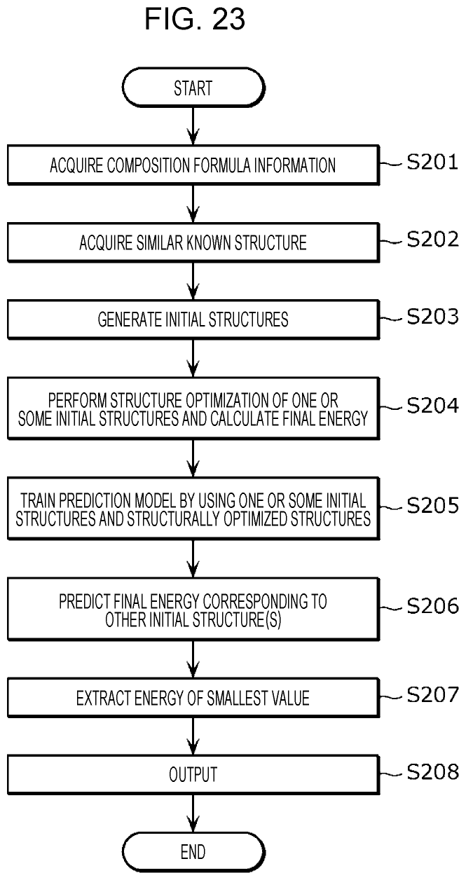

FIG. 23 is a flowchart illustrating an operation example of the search system 200 according to Embodiment 2. Processes in steps S201 to S204 and steps S206 to S208 are identical to the processes in steps S101 to S104 and S106 to S108 illustrated in FIG. 17, respectively, and therefore description thereof is omitted. That is, an overall flow of processing of the search system 200 according to Embodiment 2 is identical to that of the search system 100 according to Embodiment 1 except for step S205.

### Step S205

The training unit 205 performs machine learning of a prediction model that is a graph neural network by using, as a learning dataset, combinations of final energy calculated in step S204 and initial structures and combinations of the final energy and structurally optimized structures. The “structurally optimized structures” are intermediate structures or final structures. Then, the training unit 205 outputs the prediction model trained by the machine learning to the prediction unit 106 and the prediction model storage unit 212.

As described above, in Embodiment 2, machine learning of a prediction model is performed by further using a learning dataset including, as input data, a structurally optimized structure, that is, an intermediate structure or a final structure. Therefore, in Embodiment 2, energy corresponding to a structure obtained in a case where structure optimization is performed on an input initial structure can be predicted with higher accuracy than in Embodiment 1.

### Embodiment 3

A search system 300 (search method, or program) according to Embodiment 3 of the present disclosure is described in detail below with reference to the drawings. The search system 300 according to Embodiment 3 is different from the search system 100 according to Embodiment 1 and the search system 200 according to Embodiment 2 in that a prediction model concerning a known structure that has been trained by machine learning in advance is used to predict second energy corresponding to an atomic arrangement structure obtained in a case where structure optimization is performed on an initial structure. Note that in the present embodiment, constituent elements identical to those in Embodiment 1 or Embodiment 2 are given identical reference signs, and description thereof is omitted.

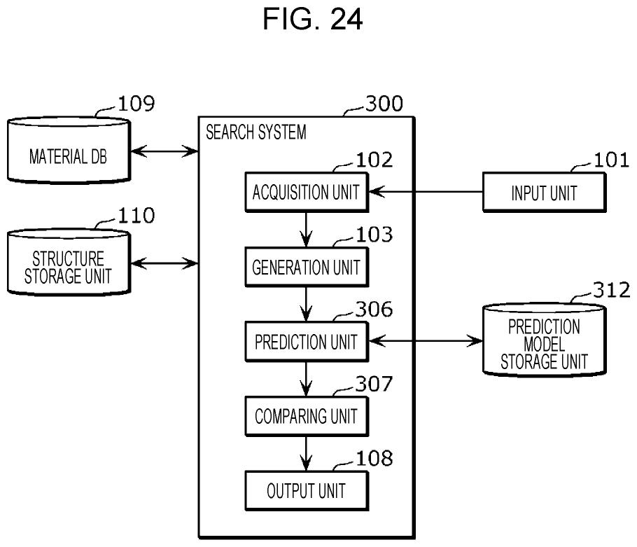

FIG. 24 is a block diagram illustrating an overall configuration including the search system 300 according to Embodiment 3. As illustrated in FIG. 24, the search system 300 includes an acquisition unit 102, a generation unit 103, a prediction unit 306, a comparing unit 307, and an output unit 108, and does not include a training unit 105 or a training unit 205. As peripheral constituents of the search system 300, an input unit 101, a material database (DB) 109, a structure storage unit 110, and a prediction model storage unit 312 are provided. Note that the peripheral constituents of the search system 300 may be included in the constituent elements of the search system 300.

Details of the constituent elements illustrated in FIG. 24 are described below. Note that constituent elements other than the prediction model storage unit 312, the prediction unit 306, and the comparing unit 307 are identical to those in Embodiment 1, and therefore description thereof is omitted.

### Prediction Model Storage Unit 312

The prediction model storage unit 312 stores therein a structure and a weight of a graph neural network concerning a prediction model that has been trained by machine learning in advance. The prediction model employed here is, for example, a prediction model concerning a known structure of a known material similar to a composition of a search target material or a prediction model trained for a general purpose. In Embodiment 3, the prediction model is the former prediction model, that is, a prediction model concerning a known structure. This prediction model is, for example, trained by machine learning in advance by using a learning dataset including a known structure as input data and including, as correct answer data, final energy corresponding to a final structure obtained by performing structure optimization on the known structure.

### Prediction Unit 306

The prediction unit 306 acquires initial structures from the structure storage unit 110. Then, the prediction unit 306 predicts final energy of the initial structures by inputting the initial structures to the trained prediction model acquired from the prediction model storage unit 312. In Embodiment 3, the prediction unit 306 predicts final energy by using the prediction model for all of the initial structures. That is, the prediction unit 306 (an eighth step) predicts energy corresponding to atomic arrangement structures obtained in a case where structure optimization is performed on the initial structures by using the prediction model for the initial structures. The “energy” is predicted values of final energy corresponding to final structures obtained in a case where structure optimization is performed on the initial structures.

The prediction unit 306 outputs the initial structures and the predicted values of final energy corresponding to the initial structures to the comparing unit 307.

### Comparing Unit 307

The comparing unit 307 acquires combinations of the initial structures and the predicted values of final energy from the prediction unit 306. Then, the comparing unit 307 generates a list in which the combinations of the initial structures and the predicted values of final energy are arranged.

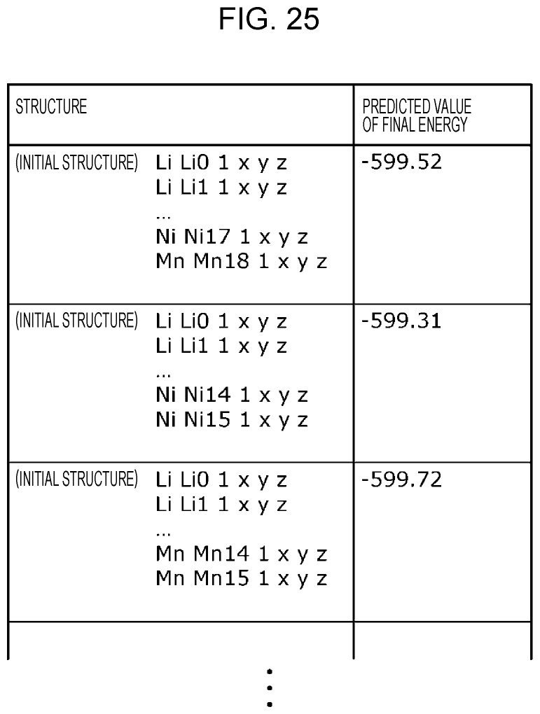

FIG. 25 illustrates an example of data generated by the comparing unit 307 according to Embodiment 3. In FIG. 25, the left column illustrates atomic arrangement of an initial structure, and the right column illustrates a predicted value of final energy corresponding to the initial structure. The comparing unit 307 sorts the predicted values of the final energy in a predetermined order on the basis of the list. In Embodiment 3, the comparing unit 307 sorts the predicted values of the final energy in an ascending order of energy values. Sorting the predicted values of the final energy corresponds to processing of extracting a smallest value, in other words, a minimum value from among the predicted values of the final energy.

That is, the comparing unit 307 (a ninth step) extracts energy of a minimum value from among the predicted values of energy. The “energy” is predicted values of final energy corresponding to final structures obtained in a case where structure optimization is performed on the initial structures. The minimum value is a smallest value among the energy.

The comparing unit 307 outputs the list in which the predicted values of the final energy are arranged as described above to the output unit 108.

### Embodiment 3: Description of Operation

Next, operation of the search system 300 is described.

### Flowchart

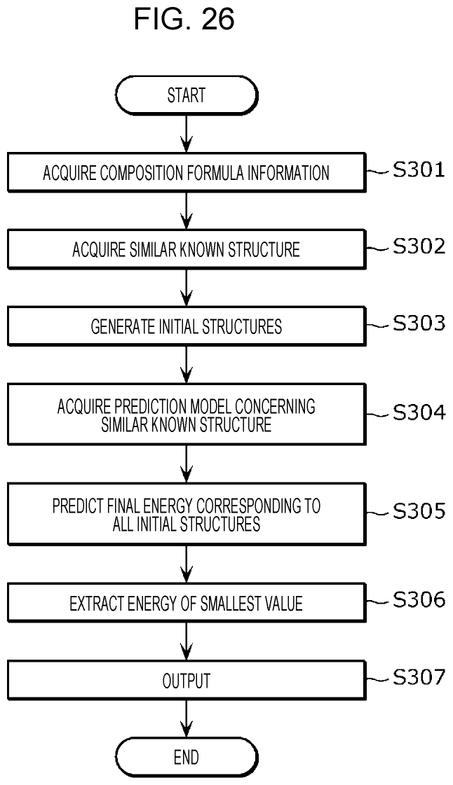

FIG. 26 is a flowchart illustrating an operation example of processing of the search system 300 according to Embodiment 3. Processes in steps S301 to S303 are identical to the processes in steps S101 to S103 illustrated in FIG. 17, respectively, and therefore description thereof is omitted.

### Step S304

The search system 300 acquires a prediction model that has been trained by machine learning in advance and is a prediction model concerning a known structure of a known material similar to a composition of a search target material and outputs the prediction model to the prediction model storage unit 312.

### Step S305

The prediction unit 306 acquires initial structures from the structure storage unit 110. Then, the prediction unit 306 calculates predicted values of final energy corresponding to the initial structures by the prediction model acquired in step S304.

### Step S306

The comparing unit 307 generates a list in which the predicted values of the final energy calculated in step S305 are sorted in an ascending order of energy values and outputs the generated list to the output unit 108. That is, the comparing unit 307 extracts energy of a smallest value from among the predicted values of the final energy.

### Step S307

The output unit 108 outputs the initial structures and the predicted values of the final energy included in the list generated in step S306 by displaying them on a display in an ascending order of energy values.

As described above, in Embodiment 3, a prediction model that has been trained by machine learning in advance is used for all initial structures, and it is therefore unnecessary to perform computation related to structure optimization. Therefore, in Embodiment 3, it is possible to search for an atomic arrangement structure considered as being thermodynamically most stable in a novel substance as in Embodiment 1 or Embodiment 2 and markedly omit computation needed for search. That is, in Embodiment 3, it is possible to reduce a computation cost and efficiently search for a stable atomic arrangement structure concerning a composition of a material as compared with a case where structure optimization is performed on one or some initial structures.

### Embodiment 4

A search system 400 (search method, or program) according to Embodiment 4 of the present disclosure is described in detail below with reference to the drawings. The search system 400 according to Embodiment 4 is different from the search system 300 according to Embodiment 3 in that a prediction model concerning a known structure that has been trained by machine learning in advance is used and whether or not the prediction model is re-trained is verified. Note that in the present embodiment, constituent elements identical to those in Embodiment 1, Embodiment 2, or Embodiment 3 are given identical reference signs, and description thereof is omitted.

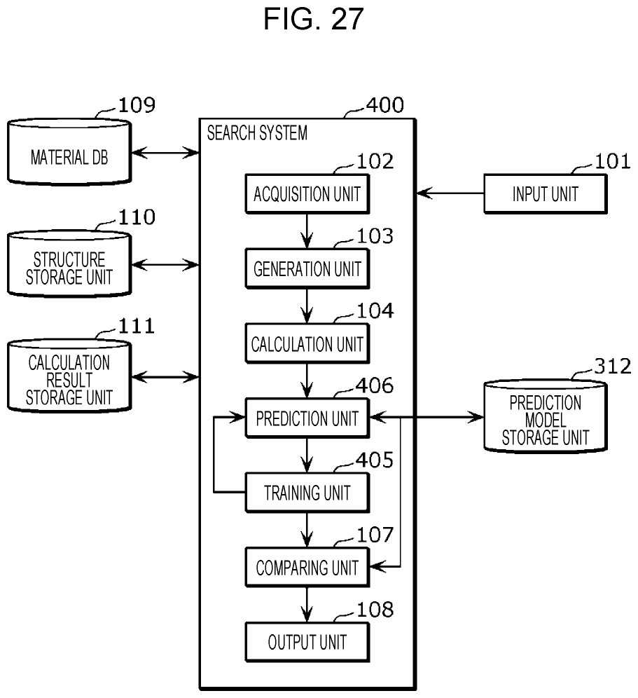

FIG. 27 is a block diagram illustrating an overall configuration including the search system 400 according to Embodiment 4. As illustrated in FIG. 27, the search system 400 includes an acquisition unit 102, a generation unit 103, a calculation unit 104, a training unit 405, a prediction unit 406, a comparing unit 107, and an output unit 108. As peripheral constituents of the search system 400, an input unit 101, a material database (DB) 109, a structure storage unit 110, a calculation result storage unit 111, and a prediction model storage unit 312 are provided. Note that the peripheral constituents of the search system 400 may be included in the constituent elements of the search system 400.

Details of the constituent elements illustrated in FIG. 27 are described below. Note that the constituent elements other than the training unit 405 and the prediction unit 406 are identical to those in Embodiment 1 or Embodiment 3, and therefore description thereof is omitted.

### Training Unit 405

The training unit 405 re-trains the prediction model in a case where the prediction unit 406 determines that prediction accuracy of a prediction model does not satisfy a condition. Specifically, the training unit 405 acquires initial structures and final energy of final structures from the calculation result storage unit 111 and re-trains the prediction model acquired from the prediction model storage unit 312 by using the initial structures and the final energy of the final structures. A learning dataset used to re-train the prediction model includes the initial structures as input data and the final energy as correct answer data.

The training unit 405 outputs the re-trained prediction model to the prediction unit 406 and the prediction model storage unit 312.

The prediction model storage unit 312 stores therein a structure and a weight of a graph neural network concerning the prediction model re-trained by the training unit 405. That is, in the prediction model storage unit 312, the prediction model that is already stored is updated to the re-trained prediction model.

### Prediction Unit 406

The prediction unit 406 acquires an initial structure and final energy of a final structure from the calculation result storage unit 111. The prediction unit 406 acquires the prediction model from the prediction model storage unit 312. The prediction model acquired by the prediction unit 406 is a prediction model that has not been re-trained yet by the training unit 405. The prediction unit 406 predicts final energy of the initial structure by inputting the initial structure to the acquired prediction model. Then, the prediction unit 406 verifies prediction accuracy of the prediction model by comparing a predicted value of the final energy and the final energy acquired from the calculation result storage unit 111. Specifically, for example, in a case where a root mean squared error (RMSE) between the final energy and the predicted value of the final energy is smaller than a certain value, the prediction unit 406 determines that prediction accuracy of the prediction model is sufficient, that is, a condition of prediction accuracy is satisfied. On the other hand, in a case where the RMSE is larger than the certain value, the prediction unit 406 determines that the prediction accuracy of the prediction model is insufficient, that is, the condition of prediction accuracy is not satisfied. The prediction unit 406 may determine that the condition of the prediction accuracy is satisfied, for example, in a case where it is predicted that an atomic arrangement structure considered as being actually most stable is a structure ranked within certain places of ranking concerning stability. Note that a method for verifying the prediction accuracy of the prediction model is not limited to the above method and may be a different method.

That is, the prediction unit 406 (a tenth step) predicts second energy corresponding to an atomic arrangement structure obtained in a case where structure optimization is performed on at least one initial structure among one or some initial structures by using the prediction model for the at least one initial structure. Here, the second energy is a predicted value of final energy corresponding to a final structure obtained in a case where structure optimization is performed on the at least one initial structure. The prediction unit 406 (an eleventh step) verifies prediction accuracy of the prediction model by comparing first energy and the second energy. Here, the first energy is final energy of a final structure corresponding to the at least one initial structure.

In a case where the condition of the prediction accuracy of the prediction model is satisfied or in a case where the prediction model is re-trained by the training unit 405, the prediction unit 406 acquires initial structure(s) for which final energy has not been calculated from the structure storage unit 110. The “initial structure(s) for which final energy has not been calculated” are structure(s) excluding the one or some initial structures from the initial structures, that is, other initial structure(s). Then, the prediction unit 406 predicts the final energy of the initial structures by inputting the initial structures to the prediction model.

That is, in a case where a result in the prediction unit 406 (the eleventh step) satisfies a predetermined condition, that is, in a case where the condition of the prediction accuracy is satisfied, the prediction unit 406 (a twelfth step) predicts second energy corresponding to atomic arrangement structures obtained in a case where structure optimization is performed on the other initial structure(s) among the initial structures by using the prediction model for the other initial structure(s). Here, the second energy is predicted values of final energy corresponding to final structures obtained in a case where structure optimization is performed on the other initial structure(s). The prediction unit 406 outputs the initial structures and the predicted values of the final energy corresponding to the initial structures to the comparing unit 107.

### Embodiment 4: Description of Operation

Next, operation of the search system 400 is described.

### Flowchart

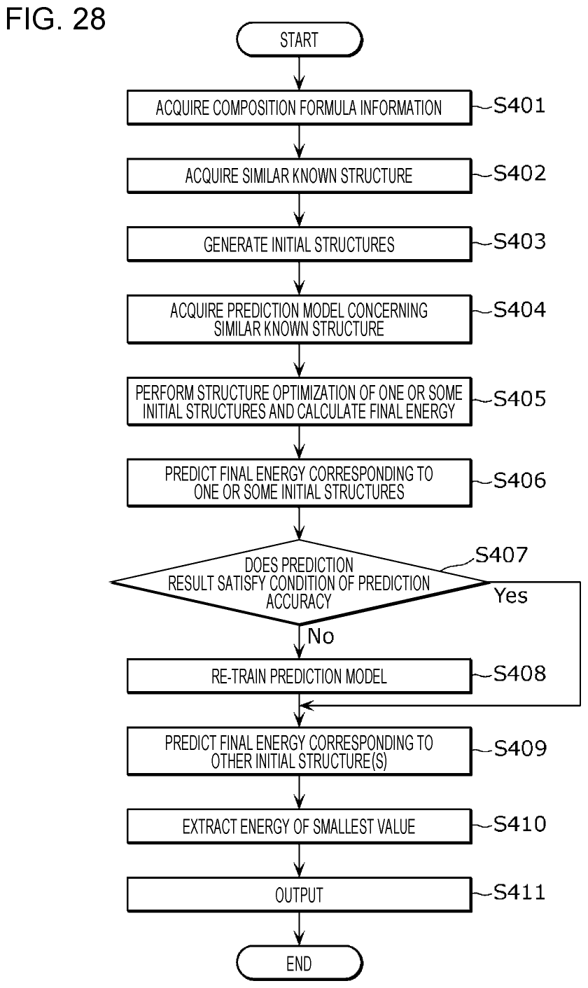

FIG. 28 is a flowchart illustrating an operation example of processing of the search system 400 according to Embodiment 4. Processes in steps S401 to S404 are identical to the processes in steps S301 to S304 illustrated in FIG. 26, respectively, and therefore description thereof is omitted.

### Step S405

The calculation unit 104 performs structure optimization on one or some initial structures among initial structures generated in step S403 and calculates final energy corresponding to final structures obtained by performing the structure optimization. Then, the calculation unit 104 outputs a calculation result to the calculation result storage unit 111.

### Step S406

The prediction unit 406 acquires the initial structures, that is, the one or some initial structures from the calculation result storage unit 111. Then, the prediction unit 406 calculates predicted values of final energy corresponding to the one or some initial structures by a prediction model acquired in step S404.

### Step S407

The prediction unit 406 verifies prediction accuracy of the prediction model by comparing the predicted values of the final energy calculated in step S406 and the final energy calculated in step S405. In a case where a prediction result satisfies a condition of the prediction accuracy (step S407: Yes), the processing proceeds to step S409. On the other hand, in a case where the prediction result does not satisfy the condition of the prediction accuracy (step S407: No), the processing proceeds to step S408.

### Step S408

The training unit 405 re-trains the prediction model that is a graph neural network by using, as a learning dataset, combinations of the final energy calculated in step S405 and the initial structures. Then, the training unit 405 outputs the re-trained prediction model to the prediction unit 406 and the prediction model storage unit 312. Note that a combination of an initial structure different from the one or some initial structures and final energy may be further used as a learning dataset to re-train the prediction model. In this case, the final energy corresponding to the different initial structure needs to be separately calculated by the calculation unit 104.

### Step S409

The prediction unit 406 acquires initial structure(s) for which final energy has not been calculated, that is, other initial structure(s) among the initial structures from the structure storage unit 110. Then, the prediction unit 406 calculates predicted values of final energy corresponding to the other initial structure(s) by the prediction model. Here, in a case where the prediction result satisfies the condition of the prediction accuracy in step S407, the prediction model acquired in S404 is used as the prediction model. On the other hand, in a case where the prediction result does not satisfy the condition of the prediction accuracy in step S407, the prediction model re-trained in step S408 is used.

### Step S410

The comparing unit 107 generates a list in which the final energy calculated in step S405 and the predicted values of the final energy calculated in step S409 are arranged in an ascending order of energy values and outputs the generated list to the output unit 108. That is, the comparing unit 107 extracts energy of a smallest value from among the final energy and the predicted values of the final energy. In other words, the comparing unit 107 (a thirteenth step) extracts third energy indicative of a minimum value on the basis of first energy and second energy. Here, the first energy is the final energy acquired from the calculation result storage unit 111, and the second energy is the predicted values of the final energy acquired from the prediction unit 406. The third energy is a minimum value among the first energy and the second energy.

### Step S411

The output unit 108 outputs the initial structures and the predicted values of the final energy included in the list generated in step S410 by displaying them on a display in an ascending order of energy values.

As described above, in Embodiment 4, a prediction model that has been trained by machine learning in advance is used, and prediction accuracy of the prediction model is verified. Therefore, in Embodiment 4, it is easy to realize a prediction model having sufficient prediction accuracy. In Embodiment 4, it is easy to more efficiently search for a stable atomic arrangement structure concerning a composition of a material by using a prediction model that satisfies a condition of prediction accuracy, that is, a prediction model of relatively high prediction accuracy.

### Modifications

Although the minimum value is a smallest value among the first energy and the second energy in each of the above embodiments, this is not restrictive. Note that the first energy is final energy calculated by the calculation unit 104, and the second energy is predicted values of final energy predicted by the prediction unit 106, 306, or 406. For example, assume that a smallest value among the first energy and the second energy is a smallest value among the second energy, the second smallest value is a smallest value among the first energy, and these values are approximate. For example, assume that a difference between the two values is within 1/10000 of the smallest value among the second energy. In this case, the minimum value may be the smallest value among the first energy instead of the smallest value among the second energy. This is because an actually calculated value is considered to be higher in accuracy than a predicted value.

Although the search systems 100 to 400 acquire initial structures generated by the generation unit 103 in each of the above embodiments, this is not restrictive. For example, in the search systems 100 to 400, initial structures generated in another system may be acquired by the acquisition unit 102. In this case, the generation unit 103 is unnecessary. That is, in the acquisition step, initial structures may be acquired by generating the initial structures or initial structures generated in another system may be acquired.

In each of the above embodiments, each constituent element may be realized by dedicated hardware or may be realized by execution of a software program suitable for the constituent element. Each constituent element may be realized in such a manner that a program executing unit such as a central processing unit (CPU) or a processor reads out a software program recorded in a recording medium such as a hard disk or a semiconductor memory and executes the software program.

Note that the following cases are also encompassed within the present disclosure.

(1) At least one of the systems is specifically a computer system that includes a microprocessor, a ROM, a RAM, a hard disk unit, a display unit, a keyboard, a mouse, and the like. A computer program is stored in the RAM or the hard disk unit. The microprocessor operates in accordance with the computer program, and thus the at least one of the systems accomplishes a function thereof. The computer program is a combination of command codes indicating a command given to a computer for accomplishment of a predetermined function.

(2) Part of or all of constituent elements that constitute at least one of the systems may include a single system large scale integration (LSI). The system LSI is a super-multi-function LSI produced by integrating constituents on a single chip and is specifically a computer system including a microprocessor, a ROM, a RAM, and the like. A computer program is stored in the RAM. The microprocessor operates in accordance with the computer program, and thus the system LSI accomplishes a function thereof.

(3) Part of or all of constituent elements that constitute at least one of the systems may include an IC card that can be detachably attached to the apparatus or a stand-alone module. The IC card or the module is a computer system that includes a microprocessor, a ROM, a RAM, and the like. The IC card or the module may include the super-multi-function LSI. The microprocessor operates in accordance with a computer program, and thus the IC card or the module accomplishes a function thereof. The IC card or the module may have tamper resistance.

(4) The present disclosure may be the methods described above. The present disclosure may be a computer program for causing a computer to realize these methods or may be a digital signal represented by the computer program.

The present disclosure may be a computer-readable recording medium, such as a flexible disc, a hard disk, a compact disc (CD)-ROM, a DVD, a DVD-ROM, a DVD-RAM, a Blu-ray (Registered Trademark) Disc (BD), or a semiconductor memory, on which the computer program or the digital signal is recorded. The present disclosure may be the digital signal recorded on such a recording medium.

The present disclosure may be the computer program or the digital signal transmitted over an electric communication line, a wireless or wired communication line, a network represented by the Internet, data broadcasting, or the like.

The program or the digital signal may be executed by another independent computer system by transporting the program or the digital signal recorded on the recording medium or transporting the program or the digital signal over the network or the like.

The present disclosure can search for a stable atomic arrangement structure without performing calculation on all atomic arrangement structure candidates and is useful for a case where a stable atomic arrangement structure of a novel material is searched for in a situation where a large-scale calculation resource cannot be prepared.

What is claimed is:

1. A search method for searching for a stable atomic arrangement structure in a three-dimensional space concerning a composition of a material, comprising causing a computer to:
acquire initial structures that are atomic arrangement structures in the three-dimensional space which the composition of the material can take;
calculate first energy corresponding to structurally optimized atomic arrangement structures by performing structure optimization on one or some initial structures among the initial structures;
predict second energy corresponding to atomic arrangement structures obtained in a case where structure optimization is performed on other initial structure(s) among the initial structures by using a prediction model for the other initial structure(s);
extract third energy indicative of a minimum value on a basis of the first energy and the second energy; and
output the third energy, a first structure, which is an atomic arrangement structure corresponding to the third energy, or the third energy and the first structure,
wherein the prediction model is trained by machine learning to output, in response to input of any atomic arrangement structure, energy corresponding to a structure obtained in a case where structure optimization is performed on the atomic arrangement structure as the second energy.

2. The search method according to claim 1, wherein
in a case where n initial structures are acquired in the acquiring, the one or some initial structures in the calculating are m initial structures, and the other initial structure(s) in the predicting are (n-m) initial structures, n being an integer of two or more and m being an integer that is equal to or more than one and is less than n.

3. The search method according to claim 1, wherein
the third energy is a smallest value among the first energy and the second energy.

4. The search method according to claim 1, wherein
in the acquiring, a known structure that is an atomic arrangement structure in a three-dimensional space of a known material similar to the composition of the material is acquired, and the initial structures are generated on a basis of the known structure.

5. The search method according to claim 4, wherein
the known material contains at least one different kind of element from an element contained in the composition of the material; and
the acquiring includes a process of substituting the different kind of element with a same kind of element as an element contained in the composition of the material.

6. The search method according to claim 4, wherein
the acquiring includes a process of expanding the known structure in at least a one-dimensional direction.

7. The search method according to claim 1, wherein
the prediction model is a model trained by machine learning by using a first learning dataset including the initial structures as input data and including the first energy corresponding to the initial structures as correct answer data.

8. The search method according to claim 7, wherein
the prediction model is a model trained by machine learning by further using a second learning dataset including the structurally optimized atomic arrangement structures as input data and including the first energy corresponding to the structurally optimized atomic arrangement structures as correct answer data.

9. The search method according to claim 1, wherein
the number of one or some initial structures in the calculating is equal to or less than 90% of the total number of initial structures.

10. A search system for searching for a stable atomic arrangement structure in a three-dimensional space concerning a composition of a material, comprising:
a generator that generates initial structures that are atomic arrangement structures in the three-dimensional space which the composition of the material can take;
a calculator that calculates first energy corresponding to structurally optimized atomic arrangement structures by performing structure optimization on one or some initial structures among the initial structures;
a predictor that predicts second energy corresponding to atomic arrangement structures obtained in a case where structure optimization is performed on other initial structure(s) among the initial structures by using a prediction model for the other initial structure(s); and
an output unit that outputs the first energy and the second energy,
wherein the prediction model is trained by machine learning to output, in response to input of any atomic arrangement structure, energy corresponding to a structure obtained in a case where structure optimization is performed on the atomic arrangement structure as the second energy.

11. The search system according to claim 10, wherein
the output unit outputs third energy indicative of a minimum value that is extracted on a basis of the first energy and the second energy, a first structure, which is an atomic arrangement structure corresponding to the third energy, or the third energy and the first structure.

12. A recording medium that is a non-volatile computer-readable recording medium storing a program for searching for a stable atomic arrangement structure in a three-dimensional space concerning a composition of a material, the program causing a computer to execute a process comprising:
acquiring initial structures that are atomic arrangement structures in the three-dimensional space which the composition of the material can take;
calculating first energy corresponding to structurally optimized atomic arrangement structures by performing structure optimization on one or some initial structures among the initial structures;
predicting second energy corresponding to atomic arrangement structures obtained in a case where structure optimization is performed on other initial structure(s) among the initial structures by using a prediction model for the other initial structure(s);
outputting the first energy and the second energy,
wherein the prediction model is trained by machine learning to output, in response to input of any atomic arrangement structure, energy corresponding to a structure obtained in a case where structure optimization is performed on the atomic arrangement structure as the second energy.

13. The recording medium according to claim 12, the program further causes the computer to execute extracting third energy indicative of a minimum value on a basis of the first energy and the second energy,
wherein in the outputting, the third energy, a first structure, which is an atomic arrangement structure corresponding to the third energy, or the third energy and the first structure are further output.

14. A prediction model construction method comprising causing a computer to:
acquire an initial structure that is an atomic arrangement structure in a three-dimensional space which a composition of a material can take; and
perform machine learning so that in response to input of any atomic arrangement structure, energy corresponding to a structure obtained in a case where structure optimization is performed on the atomic arrangement structure is output by using a learning dataset including the initial structure as input data and including, as correct answer data, energy corresponding to an atomic arrangement structure obtained by performing structure optimization on the initial structure.

15. A prediction model construction device comprising:
a generator that generates an initial structure that is an atomic arrangement structure in a three-dimensional space which a composition of a material can take; and
a trainer that performs machine learning so that in response to input of any atomic arrangement structure, energy corresponding to a structure obtained in a case where structure optimization is performed on the atomic arrangement structure is output by using a learning dataset including the initial structure as input data and including, as correct answer data, energy corresponding to an atomic arrangement structure obtained by performing structure optimization on the initial structure.

16. A search method for searching for a stable atomic arrangement structure in the three-dimensional space concerning the composition of the material by using a prediction model trained by machine learning by the prediction model construction device according to claim 15, the search method comprising causing a computer to:
acquire the initial structures;
predict energy corresponding to atomic arrangement structures obtained in a case where structure optimization is performed on the initial structures by using the prediction model for the initial structures; and
extract energy indicative of a minimum value from among predicted values of the energy.

17. A search method for searching for a stable atomic arrangement structure in the three-dimensional space concerning the composition of the material by using a prediction model trained by machine learning by the prediction model construction device according to claim 15, the search method comprising causing a computer to:
acquire the initial structures;
calculate first energy corresponding to structurally optimized atomic arrangement structures by performing structure optimization on one or some initial structures among the initial structures;
predict second energy corresponding to an atomic arrangement structure obtained in a case where structure optimization is performed on at least one of the one or some initial structures by using the prediction model for the at least one of the one or some initial structures; and
verify prediction accuracy of the prediction model by comparing the first energy and the second energy.

18. The search method according to claim 17, further comprising causing the computer to:
in a case where a result in the verifying satisfies a predetermined condition,
predict the second energy corresponding to atomic arrangement structures obtained in a case where structure optimization is performed on other initial structure(s) among the initial structures by using the prediction model for the other initial structure(s); and
extract third energy indicative of a minimum value on a basis of the first energy and the second energy.
